(19) United States
(12) Patent Application Publication
(10) Pub. No.: US 2009/0215699 A1
(43) Pub. Date: Aug. 27, 2009

(54) Pharmaceutically Active Antiviral Peptides

(75) Inventors: Gerald Bacher, Germering (DE); Karl Heinrich Wiesmüller, Tübingen (DE); Birgit Schäfer, Heidelberg (DE); Joachim Hauber, Hamburg (DE)

Correspondence Address: J C PATENTS, INC., 4 VENTURE, SUITE 250, IRVINE, CA, 92618, US

(73) Assignee: FOUNDATION FOR FATEL RARE DISEASES, Vaduz (LI)

(21) Appl. No.: 11/722,865

(22) PCT Filed: Jan. 5, 2006
(86) PCT No.: PCT/EP2006/000057

§ 371 Date: Apr. 13, 2009

Related U.S. Application Data
(60) Provisional application No. 60/641,145, filed on Jan. 5, 2005.

(51) Int. Cl. A61K 38/10; C07K 7/00

(52) U.S. Cl. 514/15; 530/328

(57) ABSTRACT

The inventive peptides were found to have strong antiviral activity against HIV in general and particularly strong drug-resistant HIV activity without exerting any toxic or antiproliferative effects on cells. Consequently, using of the inventive peptides improves the conventional HIV therapy with its toxic side effects.

The present invention relates to antiviral peptides and their use as pharmaceutically active agents, especially for prophylaxis and treatment of virally caused diseases and infections, including opportunistic infections.

### Virally Caused Diseases and Infections:

### HIV and AIDS

First, the term “AIDS” stands for Acquired Immune Deficiency Syndrome, but it has always been understood that the amount of immune deficiency we are interested in, is not a trivial one. AIDS is the name historically chosen for a new medical syndrome which is essentially 100% fatal, and thus in defining “AIDS” we are looking for people with an immune deficiency in the range which is life-threatening, and which will continue to grow relentlessly worse until life is impossible. One way to define immune deficiency is to define it by so-called “opportunistic infections”, diseases rarely seen in people whose immune system is fully functional. In the early days of AIDS, before HIV was discovered, the syndrome was indeed defined using such opportunistic diseases.

Early in the history of AIDS, it was found that the immune defect in this disease is peculiar, and that it most visibly involves a particular kind of cells in blood and lymphatic tissues (lymph nodes), called “T-lymphocytes”. In the syndrome of AIDS, certain T-lymphocytes gradually disappear from both blood and lymph tissues, and a simple T-lymphocyte count in the blood can tell how serious the reduction has been in both places (since blood lymphocytes come from the lymphatic organs). The arm of the immune system which is controlled most directly by T-lymphocytes (the body's defense against viruses and fungi) is most defective in AIDS, and viral and fungal infections are the main opportunistic infections which appear and cause death in AIDS.

After HIV was identified, statistical studies quickly picked it as the most likely answer yet proposed for the cause of AIDS. HIV has the typical lentivirus structure, identical to that of FIV and SIV. People infected with HIV were found to develop antibodies to it in the blood usually a month or two after infection, and keep them for life. The presence of antibodies in the blood for years did not seem to keep people with HIV from eventually becoming very ill, however. Today, it is known that lentiviruses are able to mutate to escape host antibodies, so that antibodies found in the same blood with these viruses often do not neutralize them, especially late in the course of disease [J. Acquir. Immune. Defic. Syndr. 7:211-219, 1994]. The HIV preferably infects CD4+ cells which get lost in AIDS patients.

Most people, however, apparently recovered completely after initial HIV infection, and felt well. As with other lentiviruses, the long latency period of HIV before secondary disease was striking. In a person with AIDS, up to 13% of lymphocytes and monocytes in the blood were found infected [N Engl J Med 326:1385-1391, 1992]. From 93% to 100% of the time, infectious virus and viral DNA could be recovered from the blood of asymptomatic people who were HIV-positive, and 100% of the time from people with AIDS [N. Engl. J. Med. 321:1621-5, 1989; AIDS 6:373-377, 1992; AIDS 8:895-900, 1994]. Higher levels of virus could be cultured from the lymphatic tissues of such people, where it was multiplying actively, even in people who appeared healthy [Clin Res 40:333, 1992; Proc Natl Acad Sci USA 88:9838-9842, 1991; J Infect Dis. 164:1051-1057, 1991; J Acquir Immune Defic Syndr 6:655-662, 1993]. Like SIV-infected monkeys and FIV-infected cats doomed to future immune failure, HIV-positive people were still infected, and most were still slowly losing immune cells in lymphatic tissues.

Opportunistic infections often also appeared, such as candida yeast infection in the throat (often the first infection), or the unusual fungal pneumonia caused by Pneumocystis carinii (this organism is particularly suppressed by CD4+ lymphocytes, and so is one of the first infections to arrive when their numbers fall). With the appearance of these markers of secondary disease, now the infected person was said to have “full-blown AIDS,” or simply, AIDS.

### Lentiviruses

The spread of lentiviruses in nature involves a complex bio-ecologic system in which host, parasite, and a range of social and environmental problems interact. The viruses are disseminated exclusively by exchange of body fluids. All factors that facilitate such exchange on a large scale may potentiate epidemics and epizootics.

Similar criteria of viral genomic organization, morphology, and biological properties let to the inclusion of human immunodeficiency virus (HIV) into the lentivirus family. Originally called lymphadenopathy-associated virus (LAV), human T-cell lymphotrophic virus III (HTLV-3), or AIDS-associated retrovirus (ARV) by the respective pioneers in the field, the name HIV was substituted by the Virus Nomenclature Committee. Subsequent new inductees into the lentivirus family, based on similar criteria, were the simian, feline, and bovine viruses. All these agents were called immunodeficiency viruses (following the precedent of HIV) irrespective of whether they caused immunodeficiency or, for that matter, any disease at all. The following table 1 gives an overview of the members of the lentivirus family known by now.

TABLE 1
Lentiviruses

| Virus | Host | Diseases |
|---|---|---|
| EIAV | Horse | anemia, wasting |
| VMV | Sheep | pneumonia, wasting, arthritis, |
| | | mastitis, encephalitis |
| CAEV | Goat | arthritis, mastitis, encephalitis |
| BIV | Cattle | None |
| FIV | Cat | immunodeficency, encephalitis, |
| | | wasting |
| SIVs | various African | None |
| | monkey species | |
| HIV-1, HIV-2 | Humans | immunodeficency, pneumonia, |
| | | encephalopathy, wasting, |
| | | gastroenteropathy, nephropathy |

SIV and FIV were discovered several years after HIV and the lesson that remains from the SIV and FIV studies is that lentiviruses alone in some circumstances are quite capable of causing slow immunosuppressive death.

FIV is called “Cat AIDS”. The feline immunodeficiency virus (FIV) is a lentivirus discovered in Petaluma, Calif. in 1986, where it was first obtained from the blood of two domestic cats living in a household in which there had been a number of deaths of cats from a strange immune deficiency disorder.

With respect to the numbers of HIV-infected individuals, each day about 16,000 people worldwide are infected with the virus, with 95% of new cases occurring in developing countries (mostly Africa). More than 2.6 million people died of AIDS in 1999 alone, the highest reported number since data gathering about the disease began in the early 1980s. According to the American Medical Association, about 45 million people worldwide are infected with HIV. Of the more than 2.6 million people died of AIDS-associated diseases in 1999, an estimated 550,000 were children under 15 years of age. Although life expectancy increased very significantly by introduction of new treatments, at the present time no cure exists for AIDS.

### Opportunistic Infections (OIs)

As stated above, the inventive peptides of the present invention and/or pharmaceutically effective salts thereof or pharmaceutical compositions containing at least one inventive peptide as an active ingredient are useful for prophylaxis and/or treatment of infections, including opportunistic infections, and diseases associated with these infections.

AIDS (acquired immune deficiency syndrome) is a condition caused by HIV. This virus attacks the immune system, the body's “security force” that fights off infections. When the immune system breaks down “opportunistic infections” (OIs) take advantage of the body's weakened defenses. Therefore, to say that someone “died of AIDS” is not entirely accurate, since it is often the opportunistic infections that cause death.

One diagnostic parameter to detect AIDS is to look at the patient's CD4+ cell counts. Another way to detect AIDS is to look for OIs: if an HIV+ individual is diagnosed with an opportunistic infection this will be taken as indication for AIDS. Thus, it is really important to develop new methods and to provide new pharmaceutically active peptides in order to prevent and treat said opportunistic infections.

Listed below are lessons about each of the major OIs & cancers that can occur during late-stage HIV disease:

Viral Infections: Cytomegalovirus (CMV), Hepatitis, Herpes Simplex Virus (oral & genital herpes), Herpes Zoster Virus (shingles), Human Papiloma Virus (HPV, genital warts, anal/cervical dysplasia/cancer), Molluscum Contagiosum, Oral Hairy Leukoplakia (OHL), Progressive Multifocal Leukoencephalopathy (PML)
Fungal Infections Aspergillosis, Candidiasis (thrush, yeast infection), Coccidioidomycosis, Cryptococcal Meningitis, Histoplasmosis
Protozoal Infections Cryptosporidiosis. Microsporidiosis, Pneumocystis Carinii Pneumonia (PCP), Toxoplasmosis

### Neurological Conditions: AIDS Dementia Complex, Peripheral Neuropathy

### Other Conditions and Complications: Apthous Ulcers, Depression & Anxiety, Fatigue and Anemia, Nausea & Diarrhea, Thrombocytopenia, Wasting Syndrome & Lipodystrophy

The past ten years of study have produced clear evidence that individuals with HIV-1 infection are at risk for both primary and secondary neurological and neurobehavioral disorders. Primary disorders occur as a result of the influence of HIV-1 on the central nervous system (CNS); whereas, secondary disorders usually occur as a result of immune system deficiencies or treatment effects. Neurobehavioral disorders associated with HIV-1 infection may be complicated by preexisting or new onset psychological and emotional disorders. Clinical research in neurological, and particularly neurobehavioral disorders associated with HIV-1 infection has been complicated by methodological issues and controversies as well as ill-defined nosology.

In the following information from current research on HIV-1 related neurological disorders, diagnosis, and treatment with focus on the AIDS related neurobehavioral disorders and a brief review of secondary HIV-related neurological disorders are presented. The inventive peptides of the present invention are also useful for prophylaxis and/or treatment of the opportunistic diseases described below.

### HIV-1 Related Neurological and Neurobehavioral Disorders

### Primary Disorders

HIV-1 can directly invade the central nervous system (CNS). Viral infection in the CNS is most often seen in mono-nuclear microglial cells and multi-nucleated giant cells. Neuronal loss is usually secondary to the presence of HIV-1 in surrounding cells and not in the neurons themselves. The process by which neuronal death occurs is speculative, although proposed mechanisms include the production of cytokines that interfere with neuronal function, production of abnormal neurotransmitter metabolites that are neurotoxic, and the presence of certain viral fragments that interfere with neurotransmitter transmission.

HIV-1 associated CNS disorders include the neurobehavioral disorders, HIV associated minor cognitive disorder and HIV associated dementia, and the neurological disorder, HIV associated myelopathy.

### HIV Associated Minor Cognitive Disorder

HIV associated minor cognitive disorder may occur in patients who are otherwise asymptomatic or mildly symptomatic. The disorder is characterized by subcortical deficits of attention, information processing speed, learning and memory, and psychomotor skills. HIV associated minor cognitive disorder may be complicated by the presence of depression or anxiety, but is not caused by psychiatric problems.

Recent studies have shown that the presence of HIV associated minor cognitive disorder increases with worsening immune function. CD4 and CD8 lymphocyte counts, CD4/CD8 ratios, and the presence of beta-2 microglobulin (B2M) in both serum and cerebrospinal fluid have been shown to correlate with severity of HIV associated cognitive disorder. However, they are not pathognomonic for the disorder.

### HIV Associated Dementia

HIV associated dementia (HAD) is a progressive disorder that initially presents as apathy, inertia, cognitive slowing, memory loss, and social withdrawal. As it progresses, multiple cognitive functions become increasingly impaired. The terminal phases are characterized by global cognitive impairment, mutism, and severe psychomotor retardation. Unlike HIV associated minor cognitive disorder, HAD rarely develops prior to constitutional problems and usually does not develop prior to other AIDS defining illnesses. As with HIV associated minor cognitive disorder, thorough neuropyschological evaluation is recommended to assist in differential diagnosis and to identify the presence of any co-existing psychiatric disturbance.

### HIV Associated Myelopathy

HIV associated myelopathy is characterized by symptoms of weakness, incoordination, and/or urinary incontinence and signs of paresis, spasticity, and hyperreflexia. This condition affects approximately 20% of adult patient with AIDS, although evidence of myelopathy is found at autopsy in 50% of patients. This condition is often associated with co-existing cognitive dysfunction.

HIV-1 has been found in the spinal cord and CSF of patients with HIV associated myelopathy; however, it is uncertain whether HIV-1 is a direct pathogen. Other conditions such as vitamin B12 deficiencies cause similar disorders, particularly in the immune-compromised patient.

### Secondary Disorders

The immuno-compromised HIV patient is at risk for numerous peripheral and central nervous system disorders that are not caused directly by the HIV virus. Peripheral nerve disorders, including sensory neuropathy, inflammatory demyelinating polyneuropathies, mononeuropathies, and cranial neuropathies are found in HIV infected patients. The incidence of neuropathies increases with worsening immune system functioning and usually occur in the presence of other HIV related disorders; however, in rare instances, peripheral neuropathies may precede other HIV symptoms. HIV-1 associated myopathy is uncommon, but may present across all stages of disease.

Immune compromised patients are at risk for neurological opportunistic infections. Cryptococcal meningitis, toxoplasmosis, cytomegalovirus (CMV), and progressive multifocal leukoencephalopathy (PML) are seen in varying incidences among AIDS patients. CMV retinitis causes a hemorrhagic retinitis in up to 20% of AIDS patients. The treatment of choice is ganciclorvir, which has demonstrated a positive clinical response in approximately 80% of patients treated.

In addition to the opportunistic infections, AIDS patients are at risk for opportunistic CNS neoplasms, metabolic encephalopathies, cerebrovascular disease, and neurosyphilis.

### REFERENCES HIV SECTION

1) Science 1983 May 20; 220(4599):868-71; Isolation of a T-lymphotrophic retrovirus from a patient at risk for acquired immune deficiency syndrome (AIDS); Barre-Sinoussi F, Chemann J C, Rey F, Nugeyre M T, Chamaret S, Gruest J, Dauguet C, Axler-Blin C, Vezinet-Brun F, Rouzioux C, Rozenbaum W, Montagnier L
2.) Science 1984 May 4; 224(4648):500-3; Frequent detection and isolation of cytopathic retroviruses (HTLV-III) from patients with AIDS and at risk for AIDS; Gallo R C, Salahuddin S Z, Popovic M, Shearer G M, Kaplan M, Haynes B F, Palker T J, Redfield R, Oleske J, Safai B, et al
3.) FASEB J 1991 July; 5(10):2361-8; Regulation of HIV-1 gene expression; Cullen B R
4.) AIDS 1998 Mar. 26; 12(5):453-60; Resistance and cross-resistance with saquinavir and other HIV protease inhibitors: theory and practice; Roberts N A, Craig J C, Sheldon J
5.) Nat Med 1998 November; 4(11):1302-7; Potent suppression of HIV-1 replication in humans by T-20, a peptide inhibitor of gp41-mediated virus entry; Kilby J M, Hopkins S, Venetta T M, DiMassimo B, Cloud G A, Lee J Y, Alldredge L, Hunter E, Lambert D, Bolognesi D, Matthews T, Johnson M R, Nowak M A, Shaw G M, Saag M S
6.) Ann Intern Med 1997 Nov. 15; 127(10):947; Severe diabetes associated with protease inhibitor therapy; Visnegarwala F, Krause K L, Musher D M
7.) AIDS 1999 Mar. 11; 13(4):465-71; Redistribution of body fat in HIV-infected women undergoing combined antiretroviral therapy; Gervasoni C, Ridolfo A L, Trifiro G, Santambrogio S, Norbiato G, Musicco M, Clerici M, Galli M, Moroni M
8.) Lancet 1998 May 2; 351(9112):1328; Severe premature coronary artery disease with protease inhibitors; Henry K, Melroe H, Huebsch J, Hermundson J, Levine C, Swensen L, Daley J
9.) Gene Ther 1994 January; 1(1):32-7; Regulated expression of a dominant negative form of Rev improves resistance to HIV replication in T cells; Liu J, Woffendin C, Yang Z Y, Nabel G J
10.) Hum Gene Ther 1995 May; 6(5):625-34; RevM10-mediated inhibition of HIV-1 replication in chronically infected T cells; Escaich S, Kalfoglou C, Plavec I, Kaushal S, Mosca J D, Bohnlein E
11.) Proc Natl Acad Sci USA 1996 Apr. 2; 93(7):2889-94; Expression of a protective gene-prolongs survival of T cells in human immunodeficiency virus-infected patients; Woffendin C, Ranga U, Yang Z, Xu L, Nabel G J

### DESCRIPTION OF THE INVENTION

Surprisingly, it was found that the antiviral peptides strongly inhibit infections induced by lentiviruses and can therefore be used for prophylaxis and treatment of virally induced infections, including opportunistic infections, and also diseases associated with such infections.

The antiviral peptides of the present invention and/or pharmaceutically active salts thereof can be used for the manufacture of a medicament for prophylaxis and/or treatment of diseases and infections, including opportunistic infections, caused by viruses.

These viruses are classified into Families, Subfamilies, Genera, and Species. The criteria used for classification are: 1) nucleic acid type and structure (linear single stranded (ss) DNA, circular double stranded (ds) DNA, linear ds DNA, positive strand RNA etc.), 2) morphology and size of the virion (naked vs enveloped icosahedral or helical nucleocapsids), and 3) viral replication strategy and gene expression.

### Families of Viruses

### DNA Viruses

1. Parvoviridae: 20 nm in diameter, naked icosahedral nucleocapsids, ssDNA (5 kb), narrow host range, replicate in rapidly dividing cells, can establish latent infections with viral DNA integrated within the host genome. Examples: Erythrovirus (human parvovirus B19), erythemia infectiosum, hemolytic anemia. Site of replication: nucleus Site of budding: none
2. Papovaviridae: 55 nm in diameter, naked icosahedral nucleocapsid, circular ds DNA (5-8 kb), replicates in the nucleus and may transform cells, persists as an episome. Examples: Papillomavirus, cause warts and associated with cervical cancer; Polyomavirus (BK and JC virus) Site of replication: nucleus Site of budding: none
3. Adenoviridae: 70 nm in diameter, naked icosahedral nucleocapsids, ds linear DNA (37 kb). Example: Adenovirus, associated with infections of the respiratory tract, eye, and intestines. Site of replication: nucleus Site of budding: none
4. Herpesviridae: enveloped icosahedral nucleocapsids of 200 nm and naked icosahedral nucleocapsids of 100 nm, linear ds DNA (150 kb), replicate in the nucleus, may establish latent infections. Examples: Herpes simplex, infection of skin, eye, and genitalia; Cytomegalovirus, congenital abnormalities; Epstein-Barr virus, infectious mononucleosis, Burkitt lymphoma, and nasopharyngeal carcinoma. Site of replication: nucleus Site of budding: nucleus
5. Poxyiridae: largest vertebrate viruses, brick-shaped about 225 nm×300 nm, linear ds DNA (130-250 kb), replicates in the cytoplasm. Example: Variola virus, smallpox. Site of replication: cytoplasm Site of budding: golgi bodies
6. Hepadnaviridae: 42 nm in diameter, enveloped icosahedral nucleocapsids, smallest of the viral genomes (3 kb), envelope (Hbs Ag or surface antigen or Dane particles) can self associate forming particles of about 22 nm, this surface antigen can be group specific, replication cycle involves reverse transcription of viral RNA to DNA, infect liver cells. Example: Hepatitis B virus, hepatitis and liver cancer. Site of replication: cytoplasm Site of budding: endoplasmic reticulum

### RNA Viruses

1. Picornaviridae: 30 nm in diameter, naked icosahedral nucleocapsid, positive strand ss RNA (8 kb), replicates in the cytoplasm. Examples: Enteroviruses (Poliovirus [poliomyelitis], Coxsackievirus B, Coxsackie virus A [striated muscles damage, meningitis, common cold, diarrhea in infants, hemorrhagic conjunctivitis], and Coxsackie virus B [primarily fatty tissue and CNS damage, upper respiratory infections]); Hepatovirus (hepatitis A virus), infectious hepatitis. Site of replication: cytoplasm Site of budding: none
2. Caliciviridae: 40 nm in diameter, naked icosahedral nucleocapsids, positive ss RNA (8 kb). Example: Norwalk agent, epidemic gastroenteritis; Hepatitis E virus, hepatitis. Site of replication: cytoplasm Site of budding: none
3. Togaviridae and Flaviviridae (Arboviruses): 40-50 nm, enveloped icosahedral nucleocapsid, positive ss RNA (11 kb-12 kb), replicate in the cytoplasm. Examples: Alphaviruses and Flaviviruses, transmitted by mosquitoes and ticks, can cause encephalitis; also the Rubella virus (family Togaviridae), is not an arbovirus; can cause severe deformities of fetuses in the first trimester of pregnancy. Site of replication: cytoplasm Site of budding: plasma membrane
4. Flaviviridae: 55 nm in diameter, enveloped icosahedral nucleocapsid, 10 kb gnome. Examples: Yellow fever virus, transmitted by mosquitoes; Hepatitis C virus, transmitted sexually and by human blood/products. Site of replication: cytoplasm Site of budding: endoplasmic reticulum
5. Coronaviridae: 120 nm in diameter, enveloped helical nucleocapsid, positive strand ss RNA (30 kb). Examples: Human coronavirus, upper respiratory infections. Site of replication: cytoplasm Site of budding: golgi bodies
6. Paramyxoviridae: 150 nm in diameter, enveloped helical nucleocapsid, negative strand ss RNA (15 kb), the envelop contains a glycoprotein with hemagglutinin/neuraminidase activity and a glycoprotein with membrane fusing activity. Examples: Parainfluenza virus, respiratory infections; Mumps virus; Morbillivirus, measles; Respiratory syncytial virus, pneumonia/bronchiolitis in infants and children, common cold. Site of replication: cytoplasm Site of budding: plasma membrane
7. Rhabdoviridae: bullet-shaped enveloped helical nucleocapsis, 180 nm×75 nm, negative strand ss RNA (15 kb), natural host not humans but disease producing when a human is infected. Examples: Vesicular stomatitis virus, lesions of the mouth; Rabies virus, fatal encephalitis. Site of replication: cytoplasm Site of budding: plasma membrane
8. Filoviridae: 80 nm in diameter as a filament of 800 nm in length, shape is very polymorphic, negative strand ss RNA (13 kb). Examples: Marburg virus, hemorrhagic fever that is usually fatal; Ebola virus, hemorrhagic fever that is usually fatal.
9. Orthomyxoviridae: 120 nm diameter, enveloped helical nucleocapsid, segmented genome of spherical negative strand RNA (seven to eight segments; 13 kb), envelop with hemagglutinin/neuraminidase activity. Examples: Influenza A, B, and C viruses, respiratory infections. Site of replication: nucleus; Site of budding: plasma membrane
10. Arenaviridae: 130 nm in diameter, enveloped helical nucleocapsid, genome of two segments of circular negative ss RNA (13 kb). Examples: Lymphocytic choriomemingitis virus, sometimes fatal meningitis; Lassa virus (natural host rodents), frequently fatal hemorrhagic fever. Site of replication: cytoplasm; Site of budding: plasma membrane
11. Bunyaviridae: 100 nm in diameter, enveloped helical nucleocapsid, genome of three negative strands of ss RNA (14-20 kb) in a circular configuration. Examples: Rift Valley fever virus, fever sometimes fatal; Hantaan virus, hemorrhagic fever sometimes fatal. Site of replication: cytoplasm Site of budding: golgi bodies.
12. Reoviridae: 75 nm in diameter, naked icosahedral symmetry with two capsid shells, genome of 10-12 segments of ds RNA (18-27 kb). Examples: Mammalian reovirus, pathogenicity not determined in humans; Colorado tick fever virus, encephalitis; Rotavirus, gastroenteritis in infants. Site of replication: cytoplasm Site of budding: none
13. Retroviridae: 100 nm in diameter, enveloped helical nucleocapsid within a perhaps icosahedral core shell, genome is diploid consisting of two identical positive strands of RNA (7-10 kb), each virion contains the enzymes reverse transcriptase, intergrase, and protease, the life cycle consists of a stage at which viral RNA is transcribed to viral DNA which can integrate within the host genome, can cause latent infections. Examples: HIV, causes AIDS; Human T-cell leukemia (lymphotrophic) virus 1 (HTLV-1), Adult T-cell leukemia; HTLV-2, hairy cell leukemia. Site of replication: nucleus Site of budding: plasma membrane

### Other Viruses and Infectious Agents

1. Hepatitis D virus (Deltavirus): replication dependent on co-infection with Hepatitis B virus, 32 nm in diameter, genome consists of circular negative ss RNA (1.7 kb) with ribozyme (ribonuclease activity).
2. Proteinaceous Infectious Particles (PRIONS)

The retroviruses may be selected from the group comprising lentiviruses and oncoretroviruses. Examples for lentiviruses are FIV, SIV, BIV, HIV-1, HIV-2, visna virus, caprine arthritis-encephalitis virus (CAEV), and equine infectious anemia virus (EIAV). Most preferably, the retrovirus represents the lentivirus HIV-1 or HIV-2. HTLV-I, HTLV-II and BLV belong to the oncoretroviruses. Furthermore, the excellent antiviral activity of the antiviral peptides can preferably be used to treat retroviruses wherein the retrovirus is a T-cell trophic HIV strain or wherein the retrovirus is a macrophage-trophic HIV strain.

Paramyxoviruses comprise respiratory syncytial virus, parainfluenza viruses, mumps virus, and measles virus. More preferably, the paramyxovirus is respiratory syncytial virus. The herpesvirus family comprises the human herpes viruses 1 to 8 and different herpes viruses for various animal species as shown below in table 2:

TABLE 2
Members of the herpesvirus family

| Subfamily | Genus | Human | Animal |
|---|---|---|---|
| Alpha- | simplex virus | human herpesvirus 1 | bovine herpesvirus 2 |
| herpesvirus | | (herpes simplex virus 1) | |
| | | human herpesvirus 2 | cercopithecine herpes- |
| | | (herpes simplex virus 2) | virus 1 , (herpes B virus) |
| | varicella virus | human herpesvirus 3 | Pseudorabiesvirus |
| | | (Varicella Zoster virus) | |
| | | | bovine herpesvirus 1 |
| | | | equine-abortion virus |
| Beta- | cytomegalovirus | human herpesvirus 5 | |
| herpesvirus | | (HCMV) | |
| | muromegalovirus | | murine herpesvirus 1 |
| | roseolovirus | human herpesvirus 6, | aotine herpesvirus 1, 3 |
| | | human herpesvirus 7 | |
| Gamma- | lymphocrytovirus | human herpesvirus 4 | cercopithecine herpes- |
| herpesvirus | | (Epstein-Barr virus) | virus 2 |
| | | | pongine herpesvirus 1 |
| | rhadinovirus | human herpesvirus 8 | ateline herpesvirus 2 |
| | | | Saimirine herpesvirus 1 |

More preferably, the herpesvirus is selected from Herpes simplex virus I, Herpes simplex virus II, Varicella Zoster virus, Epstein-Barr virus, HCMV, or HHV8. The hepadnavirus are selected from HBV, Ground-Squirrel-Hepatitis virus (GSHV), or Woodchuck hepatitis virus (WHV).

The HIV is according to Gallo and Barre-Sinoussi the causative agent of the acquired immune deficiency syndrome (AIDS) (Barre-Sinoussi et al., 1983; Gallo et al., 1984). In humans, HIV infection leads to development of immune incompetence, opportunistic infections, neurological disorders like cognitive and motor impairment, neoplastic growth, and death. HIV infection is pandemic and HIV associated diseases represent a major, increasing world health problem. Considerable effort is being put into the design of effective therapeutics, but no curative anti-retroviral drugs against AIDS exist. Therefore, design and testing of effective, non-toxic, novel anti-retroviral drugs with novel modes of action are still needed.

The virus may be selected from the retroviruses, particularly lentiviruses and oncoretroviruses (HTLV-BLV group), adenoviruses, hepadnaviruses, herpesviruses and influenza viruses. More particularly, the virus is a retrovirus which may be selected from lentiviruses such as HIV-1, HIV-2, FIV, bovine immunodeficiency virus (BIV), simian immunodeficiency viruses (SIVs), visna Maedi virus (VMV), caprine arthritis-encephalitis virus (CAEV), equine infectious anemia virus (EIAV), or oncoretroviruses such as human T-cell leukemia virus I (HTLV-I), human T-cell leukemia virus II (HTLV-II) and bovine leukemia virus (BLV). Most preferably, the retrovirus is HIV-1 or HIV-2 and the herpesvirus is Herpes simplex virus I, Herpes simplex virus II, Varicella Zoster virus, Epstein-Barr virus, HCMV, or HHV8.

The peptides of the present invention can also be used for the manufacture of an agent for prophylaxis and/or treatment of diseases and infections caused by viruses integrating or not integrating in the genome of a cell. Examples for viruses which do not integrate in the genome of a cell are paramyxoviruses. Paramyxoviruses comprise, for instance, parainfluenza viruses, mumps viruses, measles virus, and respiratory syncytial viruses. More preferably, the virus is respiratory syncytial virus.

The present invention discloses also a method wherein the inventive peptides are used to treat viruses which are resistant against most common antiviral drugs. Thereby, the peptides and/or pharmaceutically acceptable salts thereof are administered in a dosage corresponding to an effective concentration in the range of 0, 1-300 μM. More preferably, the inventive peptides of the present invention are administered in a dosage corresponding to an effective concentration in the range of 1-30 μM.

The peptides disclosed herein are especially useful for the treatment of drug-resistant HIV strains as well as multidrug-resistant HIV strains and HIV strains which are resistant against a drug combination. Said HIV strains are especially resistant against the drugs mentioned in Table 3 as well as combinations of these drugs or against the commonly used drug combinations for the treatment of HIV and AIDS.

A further aspect of the present invention relates to the use of at least one of the peptides according to general SEQ ID No. 1 in combination with at least one anti-viral drug especially at least one anti-HIV drug. A list of suitable drugs for said drug combination is given in Table 3.

Surprisingly, it was found that the inventive peptides of SEQ ID NO: 01 are potent inhibitors of HIV replication in a submillimolar, especially micromolar, range on aggressive T-cell trophic HIV strains, aggressive macrophage-trophic HIV strains, on primary HIV isolates from patents as well as on aggressive multidrug-resistant HIV strains such as HIV-1 FE9 (Hauber et al., J. Clin. Invest. 2005, 115(1), 76-85) without apparent cellular toxicity at the effective antiviral dosage.

These findings suggest that the mentioned peptides has advantages over the currently used drugs in the treatment of HIV diseases, e.g. the Highly Active Antiretroviral Therapy (HAART), consisting of Nucleoside Analog Reverse Transcriptase Inhibitors (NRTIs), Non-Nucleoside Reverse Transcriptase Inhibitors (NNRTIs) and Protease Inhibitors (PIs). This therapy is accompanied by quick emergence of resistant viral strains. In some patients, replication of resistant HIV-1 leads to emergence of more virulent variants of HIV-1, which are associated with an accelerated loss of CD4+ cells and confer a significantly increased risk of disease progression and death (D'Aquila et al., 1995). Resistance emerges as a consequence of the selective pressure of incompletely suppressive therapy and is determined by mutations in the HIV reverse transcriptase and protease genes. Primary mutations alter the binding of the drug to its target resulting in an increase in the amount of drug necessary to inhibit the enzyme. Secondary mutations increase resistance by improving the fitness of viruses carrying primary infections. In the case of NRTIs, the mutation M148V is an example of a key mutation leading to high-level resistance. Additional positions of mutations include M41L, K70R, T215Y (Zidovudine), L74V (Didanosine), T69D (Zalcitabine), Y115F (Abacavir). Multidrug resistance conferring cross-resistance to the entire NRTI class is well recognized (Q151M and insertion mutation T69SSS). Cross-resistance is also extensive among the three currently used NNRTIs Efavirenz, Nevirapine and Delavirdine which makes them inactive against the virus expressing the K103N mutation in the reverse transcriptase gene. The K103N mutation acts by inhibiting formation of the drug-binding pocket. For this class of drugs, the “first shot” is most frequently the “only shot”. Also, cross-resistance between the PIs is rather a rule than an exception (Indinavir and Ritonavir have almost identical resistance patterns—K20M, V32I, M36I, M46I, I54V, L63P, A71V, V82A/T/F, I84V, L90M—and Saquinavir-resistant strains are also cross-resistant to other PIs—L10I, K20M, I84V, L90M) (Roberts et al., 1998). Even for the novel type of anti HIV drugs like T20, a 36 amino acid peptide derived from the HIVgp41 protein which inhibits fusion of the virus to the host cell, resistance has already been demonstrated in vitro (Kilby et al., 1998). In addition, patients on HAART face long-term side effects, including pancreatitis, peripheral neuropathies, hepatotoxicity, diabetes (Visnegarwala et al., 1997), or metabolic abnormalities in body fat redistribution (Gervasoni et al., 1999), as well as in the glucose metabolism and the cardiovascular field (Henry et al., 1998). For many patients, who experience the above mentioned drug toxicities, a structured treatment interruption is an unavoidable necessity (“Drug holidays”). However, during this time the suppressed virus may bounce back.

Early receptor-mediated events leading to the entry of human immunodeficiency virus (HIV) into cells. HIV type 1 (HIV-1) and HIV-2 use similar mechanisms to gain entry into cells that involve the use of two cell surface receptors, CD4 and a chemokine receptor. The interactions of these receptors with the glycoprotein spikes on virus particles trigger the fusion of viral and cell membranes (Binley, J., and J. P. Moore. 1997. HIV-cell fusion. The viral mousetrap. Nature 387:346-348). Many details of these events are now understood at a molecular level, but later postfusion events remain largely undefined. After fusion of viral and cellular membranes, the virion core is released and disassembles in the cytoplasm by an obscure process. Once exposed to the cytoplasmic milieu, the viral reverse transcriptase (RT) transcribes the viral RNA into double-stranded DNA. The viral DNA, in addition to the matrix (MA), nucleocapsid (NC), integrase (IN), RT, and Vpr, constitutes the preintegration complex (PIC) (Stevenson, M. 2000. HIV nuclear import: what's the flap? Nat. Med. 6:626-628). In nondividing cells, access to the nucleus by the PIC is limited by the presence of the nuclear membrane and an active mechanism for nuclear importation of the PIC is needed (Lewis, P., M. Hensel, and M. Emerman. 1992 Human immunodeficiency virus infection of cells arrested in the cell cycle. EMBO J. 11:3053-3058). Viral proteins, in particular Vpr and IN and probably also MA, all of which contain nuclear localization signal motifs, act in concert with cellular proteins to mediate this transport. More recently, a positive-strand DNA “flap” produced during reverse transcription has been shown to be a new player in nuclear entry in nondividing cells. In contrast, in dividing cells access to the nucleus is gained during mitotic division, when the nuclear membrane is dissipated.

The details of postfusion events and those surrounding and supporting the reverse transcription process are for the most part unknown. Cellular division itself is not needed for reverse transcription to occur (Zack, J. A. 1995. The role of the cell cycle in HIV-1 infection. Adv. Exp. Med. Biol. 374:27-31). However, events which occur during cellular activation and cell proliferation have been demonstrated to influence reverse transcription. In nondividing and/or quiescent cells reverse transcription cannot be completed and short viral DNA transcripts result. Early studies suggest that nondividing macrophages could be productively infected by HIV-1 and thus were capable of accommodating reverse transcription. More recent studies propose that infection of macrophages in culture may result from infection of a small proliferating population, although these results are controversial. One explanation for the lack of reverse transcription in nondividing quiescent cells is that such cells have limited levels of deoxyribonucleoside triphosphates (dNTPs).

Surprisingly, the inventive peptides suppress the expression of viral proteins and subsequently the cellular generation of HIV. Previously, it has been described that these peptides inhibit directly the activity of heme oxygenase-1 and inhibit the post-transcriptionally the expression of TNFalpa (Iyer S et al. 2000, J. Biol. Chem. 275, 17051). Consequently, the primary target of these peptides is most likely a cellular protein which is required for both HIV protein expression and TNFalpha protein expression. In another aspect, fluorescence-labelled antiviral peptides accumulates in defined areas within the cell around the nucleus, which indicates a defined cellular target protein of the inventive peptide.

This cellular target protein seems to be a key regulator for replication of several viruses. Thus, a panel of different viruses can be inhibited by the inventive peptides. Furthermore, resistant formation of the viruses is prevented due to a lack of direct interaction of the peptides with the virus. This has been demonstrated by incubating HIV with concentrations of the peptides which do not fully suppress virus replication for several month. Nevertheless, the peptides retain their full ability to inhibit virus replication indicating that no resistant formation of the virus occurred.

The inventive peptides with above mentioned mode of action can be preferentially used for manufacturing of a medicament for inhibition of infections mediated by multidrug resistant viruses.

In a related aspect to the studies disclosed herein, the present invention is directed to a method for treating virally induced diseases and infections, including opportunistic infections, in a mammal, including a human, which comprises administering to the mammal an amount of at least one peptide of SEQ ID No. 1 or pharmaceutically acceptable salts thereof effective to treat virally induced infections and/or diseases. Preferably, said method is used for treating HIV-1 infections. Said virally induced diseases and infections may by cause by the viruses comprising retroviruses, adenoviruses, hepadnaviruses, herpesviruses, influenza viruses, and paramyxoviruses.

Hepadnaviruses may be selected from the group comprising orthohepadnaviruses and avihepadnaviruses. Examples for orthohepadnaviruses are HBV, Ground-Squirrel-Hepatitis virus (GSHV), Woodchuck Hepatitis virus (WHV). Most preferably, the hepadnavirus represents the human Hepatitis B virus (HBV).

Herpesviruses may be selected from the group comprising α-herpesviruses (Simplexvirus, Varicellavirus), β-herpesviruses (Cytomegalovirus also known as human herpesvirus 5, Muromegalovirus, Roseolovirus), or γ-herpesviruses (Lymphocryptovirus, Rhadinovirus). Examples for α-herpesviruses are Herpes simplex virus type 1 (human herpesvirus 1), Herpes simplex virus type 2 (human herpesvirus 2), Varicella Zoster virus (human herpesvirus 3). Examples for γ-herpesviruses are Epstein-Barr virus (human herpesvirus 4) or human herpesvirus type 8 (HHV8). More preferably, the herpesvirus is Herpes simplex virus type 1, or Varicella Zoster virus, or Epstein-Barr virus (EBV), or human cytomegalovirus (HCMV), or human herpesvirus 6, or human herpesvirus 7, or human herpesvirus type 8 (HHV8). Most preferably, the herpesvirus represents the α-herpesviruses Herpes simplex virus type 1, or Varicella Zoster virus, or the γ-herpesviruses Epstein-Barr virus, or Human Herpes virus type 8.

Paramyxoviruses may be selected from the group comprising paramyxovirinae or pneumovirinae. Most preferably, the paramyxovirus represents the respiratory syncytial virus (RSV).

In addition thereto, a method of inhibiting nuclear export for the prevention or treatment of infectious diseases, particularly viral infections comprising administering a subject in need thereof a pharmaceutically effective amount of at least one peptide of SEQ ID No. 1 and/or pharmaceutically active salts thereof. Said infectious diseases, such as HIV-1 infections, or Hepatitis B virus infections, or Herpes-simplex-Virus 1 infections, or Epstein-Barr virus infections, or Human herpesvirus 8 infections, or Varicella-zoster virus infections, or Adenovirus infections, or Respiratory syncytial virus infections, are, for instance, diseases caused by viruses, especially lentiviruses or oncoretroviruses, hepadnaviruses, paramyxoviruses, adenoviruses, herpesviruses and influenza viruses. More preferably, said diseases are caused by the lentiviruses HIV-1 or HIV-2, or by the hepadnavirus Hepatitis B virus. The retrovirus may also be a T-cell trophic HIV strain, a monocyte-trophic HIV strain and most preferably a drug resistant virus strain. The HIV-1, or HIV-2 strain, or HBV strain may also be resistant against protease inhibitors and/or reverse transcriptase inhibitors. Thus, another aspect of the present invention is related to the use of the peptides of SEQ ID No. 1 and a method for treatment of virally induced infections and diseases, including opportunistic infections, wherein the retrovirus is a HIV-1 or HIV-2 strain which is resistant against protease inhibitors and/or reverse transcriptase inhibitors.

In order to treat said virally induced infections and diseases associated thereto at least one peptide of SEQ ID No:1 and/or pharmaceutically effective salts thereof are administered to an individual in need according to the disclosed method in a dosage corresponding to an effective concentration in the range of 0.1-300 μM, more preferably in the range of 1-30 μM, most preferably 5-20 μM. Furthermore, the peptides may be administered directly or in combination with further therapeutic peptides, especially with further antiviral agents. A list of suitable antiviral agents is shown in table 3. In relation to the above statements, the use of the peptides of the present invention and a method for the use of the inventive peptides and/or pharmaceutically active salts of said peptides is disclosed wherein at least peptide of SEQ ID No. 1 and/or pharmaceutically active salts thereof is administered in combination with further therapeutic peptides, especially with further antiviral agents. Said further antiviral agents may be selected from the drugs listed below in table 3.

Thus, the inventive peptides described in the present invention can be used in a monotherapy directly or in form of pharmaceutically acceptable compositions in order to treat virally induced infections and/or diseases. Said diseases are preferably caused by or associated with HIV-1, HIV-2, HTLV-I, HBV. Furthermore, the inventive peptides can be used as inhibitors of HIV strains with tropism for monocytes and for strains with tropism for T cells. In addition, the peptides described in the present invention can be used in combination with other antiviral agents or drugs in order to combat HIV-1, HIV-2, HTLV-I, HBV as well as for treating diseases associated with those viruses.

The inventive peptides described in the present invention can be specifically combined with NRTIs and NNRTIs of HIV-1 and HIV-2 like: AZT (Zidovudine), 3TC (Lamivudine), ddI (Didanosine), ddC (Zalcitabine), ABC (Abacavir), d4T (Stavudine), FTC (2′deoxy-5-fluoro-3′thiacidin), Emivirine, EFV (Efavirenz), DLV (Delavirdine), NVP (Nevirapine), Adefovir dipivoxil, PMPA; with PIs Indinavir, Ritonavir, Saquinavir, Nelfinavir, Amprenavir, ABT378, BMS 232632, Tipranavir, L-756,423, DMP-450, AG-1776; with Hydroxycarbamid, Mycophenolat-mofetil; with fusion inhibitors T-20, T-1249; with CXCR4 antagonists like AMD 3100, T22, ALX40-4C, NSC 651016; with CCR5 antagonists like RANTES, APO-RANTES, NSC 651016; with Hydroxyurea; with Integrase inhibitors like antraquinones, quinalizarin, L-chicoric acid, dicaffeoylquinic acid; with Zinc Finger inhibitors like dithiane compounds; with immunomodulators like Interleukin-2, Interferon-alpha, Interferon-beta, GM-CSF, G-CSF; with therapeutical vaccine strategies including live, attenuated and replication incompetent virus; killed, inactivated virus; envelope subunit protein; core subunit protein; peptides; nucleic acids of the respective retroviruses, specifically of HIV-1 or HIV-2; with antiretroviral gene-therapy approaches like antisense or dominant-negative Rev mutants. This invention also relates to the combination of the inventive peptides described in the present invention with at least one of the above mentioned drugs. The inventive peptides can be used as inhibitors of HIV-1, HIV-2, HTLV-I, HBV as well as for treating diseases associated with those viruses, where the above mentioned antiviral agents and drugs, especially NRTIs, NNRTIs or PIs, caused viral resistances against said antiviral agents or drugs, respectively. The antiviral peptides of the present invention or pharmaceutically effective salts thereof are preferably administered in a dosage corresponding to an effective concentration in the range of 0.1-300 μM, more preferably in the range of 1-30 μM, and most preferably in the range of 5-20 μM.

As mentioned above, a preferred embodiment of the present invention relates to the use of the antiviral peptides of SEQ ID No. 1 in combination with further antiviral agents. Table 3 represents a collection of suitable antiviral HIV agents which may be used in combination with at least one peptide of SEQ ID No:1 and/or pharmaceutically active salts thereof.

TABLE 3
Alphabetical List of Drugs for HIV

| Drug Name | Pharmaceutical Company |
|---|---|
| (+)-calanolide A | Sarawak Medichem |
| 3TC, Epivir ® brand lamivudine | Glaxo Wellcome |
| abacavir generic Ziagen ™, ABC, or 1592U89 | Glaxo Wellcome |
| ABC, Ziagen ™ brand abacavir, or 1592U89 | Glaxo Wellcome |
| ABT-378/r, or Kaletra ™ brand lopinavir | Abbott Laboratories |
| AG-1549, S-1153, or capravirine (CPV) | Agouron Pharmaceuticals |
| AG1661, Remune ™ brand HIV-1 Immunogen, or | Agouron Pharmaceuticals |
| Salk vaccine | |
| Aaenerase ™ brand amprenavir (APV), 141W94, or | Glaxo Wellcome |
| VX-478 | |
| aldesleukin generic Proleukin ®, or Interleukin-2 (IL- | Chiron Corporation |
| 2) | |
| amprenavir generic Agenerase ™, APV, 141W94, or | Glaxo Wellcome |
| VX-478 | |
| APV, Agenerase ™ brand amprenavir, 141W94, or | Glaxo Wellcome |
| VX-478 | |
| AZT, Retrovir ® brand zidovudine (ZDV) | Glaxo Wellcome |
| BCH-10652, or dOTC | BioChem Pharma |
| Bis(POC) PMPA, tenofovir disoproxil fumarate | Gilead Sciences |
| (TDF), or GS-902 | |
| BMS-232632 | Bristol-Myers Squibb |
| capravirine (CPV), AG-1549, or S-1153 | Agouron Pharmaceuticals |
| Coactinon® brand emivirine (EMV), or MKC-442 | Triangle Pharmaceuticals |
| Combivir ® brand zidovudine + lamivudine, or AZT + | Glaxo Wellcome |
| 3TC | |
| Coviracil ™ brand emtricitabine, or FTC | Triangle Pharmaceuticals |
| CPV (capravirine), AG-1549, or S-1153 | Agouron Pharmaceuticals |
| Crixivan ® brand indinavir (IDV), or MK-639 | Merck & Co. |
| d4T, Zerit ® brand stavudine, or BMY-27857 | Bristol-Myers Squibb |
| DAPD | Triangle Pharmaceuticals |
| ddC, or Hivid ® brand zalcitabine | Roche Laboratories |
| ddl, Videx ® brand didanosine, or BMY-40900 | Bristol-Myers Squibb |
| delavirdine generic Rescriptor ®, DLV, or U- | Agouron Pharmaceuticals |
| 90152S/T | |
| didanosine generic Videx ®, ddl, or BMY-40900 | Bristol-Myers Squibb |
| DLV, Rescriptor ® brand delavirdine, or U-90152S/T | Agouron Pharmaceuticals |
| DMP-450 | Triangle Pharmaceuticals |
| dOTC, or BCH-10652 | BioChem Pharma |
| Droxia ® brand hydroxyurea (HU) | Bristol-Myers Squibb |
| efavirenz generic Sustiva ™, EFV, or DMP-266 | DuPont Pharmaceuticals |
| EFV, Sustiva ™ brand efavirenz, or DMP-266 | DuPont Pharmaceuticals |
| emivirine generic Coactinon®,EMV, or MKC-442 | Triangle Pharmaceuticals |
| emtricitabine generic Coviracil ™, or FTC | Triangle Pharmaceuticals |
| EMV, Coactinon® brand emivirine, or MKC-442 | Triangle Pharmaceuticals |
| Epivir ® brand lamivudine, or 3TC | Glaxo Wellcome |
| epoetin alfa (erythropoietin) generic Procrit ® | Ortho Biotech |
| erythropoietin (epoetin alfa) generic Procrit ® | Ortho Biotech |
| Fortovase ® brand saquinavir (Soft Gel Cap), or SQV | Roche Laboratories |
| (SGC) | |
| FTC, or Coviracil ™ brand emtricitabine | Triangle Pharmaceuticals |
| GS-902, tenofovir disoproxil fumarate (TDF), or | Gilead Sciences |
| Bis(POC) PMPA | |
| GW-420867X | Glaxo Wellcome |
| GW-433908, or VX-175 | Glaxo Wellcome |
| HIV-1 Immunogen generic Remune ™, Salk vaccine, | Agouron Pharmaceuticals |
| or AG1661 | |
| Hivid ® brand zalcitabine, or ddC | Roche Laboratories |
| HU, or Droxia ® brand hydroxyurea | Bristol-Myers Squibb |
| hydroxyurea generic Droxia ®, or HU | Bristol-Myers Squibb |
| IDV, Crixivan ® brand indinavir, or MK-639 | Merck & Co. |
| IL-2 (Interleukin-2), or Proleukin ®^{brand} aldesleukin | Chiron Corporation |
| indinavir generic Crixivan ®, IDV, or MK-639 | Merck & Co. |
| Interleukin-2 (IL-2), or Proleukin ®^{brand} aldesleukin | Chiron Corporation |
| Invirase ® brand saquinavir (Hard Gel Cap), SQV | Roche Laboratories |
| (HGC), or Ro-31-8959 | |
| Kaletra ™ brand lopinavir, or ABT-378/r | Abbott Laboratories |
| lamivudine generic Epivir ®, or 3TC | Glaxo Wellcome |
| lopinavir generic Kaletra ™, or ABT-378/r | Abbott Laboratories |
| MKC-442, Coactinon® brand emivirine (EMV) | Triangle Pharmaceuticals |
| nelfinavir generic Viracept ®, NFV, or AG-1343 | Agouron Pharmaceuticals |
| nevirapine generic Viramune ®, NVP, or BI-RG-587 | Boehringer Ingelheim |
| NFV, Viracept ® brand nelfinavir, or AG-1343 | Agouron Pharmaceuticals |
| Norvir ® brand ritonavir (RTV), or ABT-538 | Abbott Laboratories |
| NVP, Viramune ® brand nevirapine, or BI-RG-587 | Boehringer Ingelheim |
| PNU-140690, or tipranavir | Boehringer Ingelheim |
| Procrit ® brand epoetin alfa (erythropoietin) | Ortho Biotech |
| Proleukin®^{brand} aldesleukin, or Interleukin-2 (IL-2) | Chiron Corporation |
| Remune ™ brand HIV-1 Immunogen, or Salk | Agouron Pharmaceuticals |
| vaccine | |
| Rescriptor ® brand delavirdine (DLV), or U-90152S/T | Agouron Pharmaceuticals |
| Retrovir ® brand zidovudine (ZDV), or AZT | Glaxo Wellcome |
| ritonavir generic Norvir ®, RTV, or ABT-538 | Abbott Laboratories |
| RTV, Norvir ® brand ritonavir, or ABT-538 | Abbott Laboratories |
| Salk vaccine, Remune ™ brand HIV-1 Immunogen, | Agouron Pharmaceuticals |
| or AG1661 | |
| saquinavir (Hard Gel Cap) generic Invirase ®, SQV | Roche Laboratories |
| (HGC), or Ro-31-8959 | |
| saquinavir (Soft Gel Cap) generic Fortovase ®, or | Roche Laboratories |
| SQV (SGC) | |
| Serostim ® brand somatropin | Serono Laboratories |
| somatropin generic Serostim ® | Serono Laboratories |
| SQV (HGC), Invirase ® brand saquinavir (Hard Gel | Roche Laboratories |
| Cap), or Ro-31-8959 | |
| SQV (SGC), or Fortovase ® brand saquinavir (Soft | Roche Laboratories |
| Gel Cap) | |
| stavudine generic Zerit ®, d4T, or BMY-27857 | Bristol-Myers Squibb |
| Sustiva ™ brand efavirenz (EFV), or DMP-266 | DuPont Pharmaceuticals |
| T-20 | Trimeris |
| TDF, tenofovir disoproxil fumarate, Bis(POC) PMPA, | Gilead Sciences |
| or GS-902 | |
| tenofovir disoproxil fumarate (TDF), Bis(POC) | Gilead Sciences |
| PMPA, or GS-902 | |
| tipranavir, or PNU-140690 | Boehringer Ingelheim |
| Trizivir ™ brand abacavir + zidovudine + lamivudine | Glaxo Wellcome |
| (ABC + AZT + 3TC) | |
| Videx ® brand didanosine, ddl, or BMY-40900 | Bristol-Myers Squibb |
| Videx ® EC brand didanosine (ddl): delayed-release | Bristol-Myers Squibb |
| capsules | |
| Viracept ® brand nelfinavir (NFV), or AG-1343 | Agouron Pharmaceuticals |
| Viramune ® brand nevirapine (NVP), or BI-RG-587 | Boehringer Ingelheim |
| VX-175, or GW-433908 | Glaxo Wellcome |
| zalcitabine generic Hivid ®, or ddC | Roche Laboratories |
| ZDV, Retrovir ® brand zidovudine, or AZT | Glaxo Wellcome |
| Zerit ® brand stavudine, d4T, or BMY-27857 | Bristol-Myers Squibb |
| Ziagen ™ brand abacavir (ABC), or 1592U89 | Glaxo Wellcome |
| zidovudine generic Retrovir ®, AZT, or ZDV | Glaxo Wellcome |

(Experimental drugs are italicized, and approved drugs are in regular, non-italicized type)

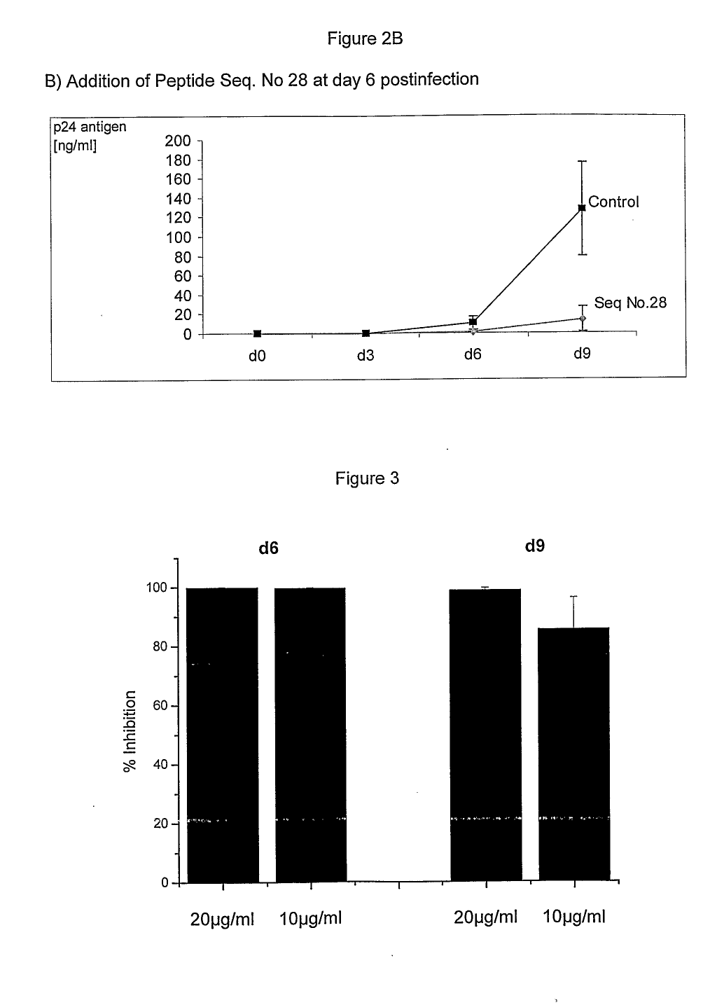

As disclosed in U.S. patent applications U.S. Ser. No. 08/838,916 and U.S. Ser. No. 09/028,083 and in the relevant literature, including Grassy et al. peptides used herein had previously been found to have properties of inhibiting T cell activity (see, e.g., Buelow et al.). In addition, peptides used herein were described in the Patent Application PCT/US0302275 as immunomodulatory in HIV infections. This means that the peptides were used to reconstitute the immune system which is affected by HIV infection as described above and especially were used in improved compositions and methods capable of accelerating and enhancing the immune reconstitution of infected individuals, and effectively treating gastrointestinal complications resulting from HIV infection. This gastrointestinal complications are normalization in gut-associated lymphoid tissue (GALT), and to alleviate the gastrointestinal abnormalities and dysfunction resulting from HIV infection. However, no significant antiviral activity of the peptides could be shown when using these peptides either alone or in combination with known antiviral agents. This was shown in FIGS. 3 and 8 of said patent application by determining the viral load of SIV-infected Rhesus macaques after treatment with either the peptide alone or in combination with a known antiviral compound.

Surprisingly we found that these peptides (Seq ID NO:01) alone have strong antiviral activity in general and particularly strong anti-HIV activity without exerting any toxic or antiproliferative effects on cells.

Generally, the phrase “antiviral peptides” as used herein is meant to encompass all of the foregoing peptides, as well as analogs, derivatives, fusion proteins and the like. In the preferred embodiment, the core sequence of the antiviral peptide desirably comprises two basic amino acids separated by from three to four hydrophobic amino acids, particularly three hydrophobic amino acids, and particularly where the N-terminus is a basic amino acid. More desirably, the C-terminal amino acid is an aromatic amino acid, particularly tyrosine. Of particular interest is where at least one of the peptide core terminal amino acids is an peptide terminal amino acid, which may be in the monomeric or oligomeric form of the compound.

More particularly, the preferred antiviral peptides for use in the compositions and methods of the present invention comprise peptides having the sequence B-X¹-X²-X³-B′-X⁴-X⁵-X⁶-J-Tyr (Seq ID NO:01), where B and B′ are independently of each other a basic amino acid, preferably Lys or Arg, particularly Arg on at least one position, preferably at both positions; J is preferably Gly, Lys or Arg or the D-isomer thereof or an aliphatic hydrophobic amino acid of from 5 to 6 carbon atoms, particularly Gly, Lys or Arg; and X¹-X⁶ are independently of each other aliphatic or aromatic amino acids. In one embodiment, at least three X amino acid residues are the same non-polar aliphatic amino acid, preferably at least four are the same non-polar aliphatic amino acid, more preferably at least five are the same non-polar aliphatic amino acid, and most preferably, all are the same non-polar aliphatic amino acid. In a preferred embodiment, the non-polar aliphatic amino acids are of from 5 to 6 carbon atoms, particularly 6 carbon atoms, particularly the non-polar aliphatic amino acids Val, Ile, Leu, and nL. Thus, in some embodiments, X¹-X⁶ are any amino acids other than a charged aliphatic amino acid, and preferably any amino acid other than a polar aliphatic amino acid.

Of the six amino acids indicated by X in the B-X¹-X²-X³-B′-X⁴-X⁵-X⁶-J-Tyr peptide sequence, preferably at least 3 are aliphatic amino acids of from 5 to 6 carbon atoms, more preferably at least 4 are aliphatic amino acids of from 5 to 6 carbon atoms, most preferably at least 5 are aliphatic amino acids of 5-6 carbon atoms, more particularly 6 carbon atoms. In a preferred embodiment, the aliphatic amino acids are non-polar aliphatic amino acids of from 5 to 6 carbon atoms, particularly Val, Ile, Leu, and nL. The other amino acids may be other uncharged aliphatic amino acids, particularly non-polar aliphatic amino acids or aromatic amino acids.

Preferred is also the use of peptides wherein B and B′ are both Arg. Furthermore such peptides are preferred wherein at least three of said X¹-X⁶ amino acid residues are the same non-polar aliphatic amino acid, preferably at least four are the same non-polar aliphatic amino acid, more preferably at least five are the same non-polar aliphatic amino acid, and most preferably, all are the same non-polar aliphatic amino acid. Said non-polar aliphatic amino acids are preferably the amino acids Val, Ile, Leu, or nL.

Compositions of particular interest will have the following formula: Arg-B-U-X²-X³-Arg-X⁴-X⁵-X⁶-J-Tyr, especially Arg-U-X-X-Arg-X-X-X-J-Tyr (Seq ID NO: 02) wherein all of the symbols have been defined previously except U, which comprises an uncharged aliphatic amino acid or aromatic amino acid, particularly a non-polar aliphatic amino acid or aromatic amino acid.

The amino acids may be naturally occurring amino acids or D-isomers thereof. The peptides may have one or more D-stereoisomer amino acids, up to all of the amino acids. Additionally, the antiviral peptides may comprise oligomers of the subject peptides, particularly dimers thereof, or comprise a cyclic peptide, that is a ring structure, as further described below.

For the purposes of this invention, the amino acids (for the most part natural amino acids or their D-stereoisomers) will be broken down into the following categories:

### 1. Aliphatic

(a) non-polar aliphatic: Gly, Ala, Val, nL, Ile, Leu
(b) polar aliphatic:
   (1) uncharged: Cys, Met, Ser, Thr, Asn, Gln
   (2) charged: Asp, Glu, Lys, Arg

### 2. Aromatic:

### Phe, His, Trp, Tyr

wherein Pro may be included in the non-polar aliphatic amino acids, but will normally not be included.

“nL” represents norleucine, where the non-polar aliphatic amino acids may be substituted with other isomers.

More preferably, peptides having the amino acid compositions as follows:

(Seq ID NO: 03)

| | |
|---|---|
| 1 | Arg Leu Leu Leu Arg Leu Leu Leu Gly Tyr |

(Seq ID NO: 04)

| | |
|---|---|
| 2 | Arg Val Leu Leu Arg Leu Leu Leu Gly Tyr |

(Seq ID NO: 05)

| | |
|---|---|
| 3 | Arg He Leu Leu Arg Leu Leu Leu Gly Tyr |

(Seq ID NO: 06)

| | |
|---|---|
| 4 | Arg Leu Val Leu Arg Leu Leu Leu Gly Tyr |

(Seq ID NO: 07)

| | |
|---|---|
| 5 | Arg Leu Ile Leu Arg Leu Leu Leu Gly Tyr |

(Seq ID NO: 08)

| | |
|---|---|
| 6 | Arg Leu Leu Val Arg Leu Leu Leu Gly Tyr |

(Seq ID NO: 09)

| | |
|---|---|
| 7 | Arg Leu Leu Ile Arg Leu Leu Leu Gly Tyr |

(Seq ID NO: 10)

| | |
|---|---|
| 8 | Arg Leu Leu Leu Arg Val Leu Leu Gly Tyr |

(Seq ID NO: 11)

| | |
|---|---|
| 9 | Arg Leu Leu Leu Arg Ile Leu Leu Gly Tyr |

(Seq ID NO: 12)

| | |
|---|---|
| 10 | Arg Leu Leu Leu Arg Leu Val Leu Gly Tyr |

(Seq ID NO: 13)

| | |
|---|---|
| 11 | Arg Leu Leu Leu Arg Leu Ile Leu Gly Tyr |

(Seq ID NO: 14)

| | |
|---|---|
| 12 | Arg Leu Leu Leu Arg Leu Leu Val Gly Tyr |

(Seq ID NO: 15)

| | |
|---|---|
| 13 | Arg Leu Leu Leu Arg Leu Leu Ile Gly Tyr |

(Seq ID NO: 16)

| | |
|---|---|
| 14 | Arg Trp Leu Leu Arg Leu Leu Leu Gly Tyr |

(Seq ID NO: 17)

| | |
|---|---|
| 15 | Arg Leu Trp Leu Arg Leu Leu Leu Gly Tyr |

(Seq ID NO: 18)

| | |
|---|---|
| 16 | Arg Leu Leu Trp Arg Leu Leu Leu Gly Tyr |

(Seq ID NO: 19)

| | |
|---|---|
| 17 | Arg Leu Leu Leu Arg Trp Leu Leu Gly Tyr |

(Seq ID NO: 20)

| | |
|---|---|
| 18 | Arg Leu Leu Leu Arg Leu Trp Leu Gly Tyr |

(Seq ID NO: 21)

| | |
|---|---|
| 19 | Arg Leu Leu Leu Arg Leu Leu Trp Gly Tyr |

(Seq ID NO: 22)

| | |
|---|---|
| 20 | Arg Tyr Leu Leu Arg Leu Leu Leu Gly Tyr |

(Seq ID NO: 23)

| | |
|---|---|
| 21 | Arg Leu Tyr Leu Arg Leu Leu Leu Gly Tyr |

(Seq ID NO: 24)

| | |
|---|---|
| 22 | Arg Leu Leu Tyr Arg Leu Leu Leu Gly Tyr |

(Seq ID NO: 25)

| | |
|---|---|
| 23 | Arg Leu Leu Leu Arg Tyr Leu Leu Gly Tyr |

(Seq ID NO: 26)

| | |
|---|---|
| 24 | Arg Leu Leu Leu Arg Leu Tyr Leu Gly Tyr |

(Seq ID NO: 27)

| | |
|---|---|
| 25 | Arg Leu Leu Leu Arg Leu Leu Tyr Gly Tyr |

(Seq ID NO: 28)

| | |
|---|---|
| 26 | Arg nL nL nL Arg nL nL nL Gly Tyr |

nL = norleucine

Either or both the N- and C-terminus of the peptide may be extended by not more than a total of about 100, usually not more than a total of about 30, more usually not more than about 20 amino acids, often not more than about 9 amino acids, where the amino acids will have fewer than 25%, more usually fewer than 20% polar amino acids, more particularly, fewer than 20% which are charged amino acids. Thus, extensions of the above sequences in either direction are mainly done with lipophilic, uncharged amino acids, particularly non-polar aliphatic amino acids and aromatic amino acids. The peptides may comprise L-amino acids, D-amino acids, or mixtures of D and L amino acids. Exceptions to the number of amino acid extensions are contemplated when the peptides are expressed as fusion or chimeric proteins, as described below.

The peptides may be in the form of oligomers, particularly dimers of the peptides, which may be head to head, tail to tail, or head to tail, there being not more than about 6 repeats of the peptide. The oligomer may contain one or more D-stereoisomer amino acids, up to all of the amino acids. The oligomers may or may not include linker sequences between the peptides. When linker sequences are used, suitable linkers include those comprising uncharged amino acids and (Gly) n, where n is 1-7, Gly-Ser (e.g., (GS) n, (GSGGS) n and (GGGS) n, where n is at least 1), Gly-Ala, Ala-Ser, or other flexible linkers, as known in the art. Linkers of Gly or Gly-Ser may be used since these amino acids are relatively unstructured, which allows interaction of individual peptides with cellular target molecules and limits structural perturbations between peptides of the oligomer.

Antiviral peptides may be in a structurally constrained form such as cyclic peptides of from about 9-50, usually 12 to 36 amino acids, where amino acids other than the specified amino acids may be present as a bridge. Thus, for example, addition of terminal cysteines allows formation of disulfide bridges to form a ring peptide. In some instances, one may use other than amino acids to cyclize the peptide. Bifunctional crosslinking agents are useful in linking two or more amino acids of the peptide. Other methods for ring formation are described in Chen et al., Proc. Natl. Acad. Sci. USA 89: 5872-5876 (1992); Wu et al., Protein Engineering 6: 471-478 (1993); Anwer, et al., Inf. J. Pep. Protein Res. 36: 392-399 (1990); and Rivera-Baeza, et al. Neuropeptides 30: 327-333 (1996); all references incorporated by reference. Alternatively, structurally constrained peptides are made by addition of dimerization sequences to the N- and C-terminal ends of the peptide, where interaction between dimerization sequences lead to formation of a cyclic type structure. In other instances, the subject peptides are expressed as fusions to other proteins, which provide a scaffold for constrained display on a surface exposed structure, such as a loop of a coiled-coil or p-turn structure.

One or both, usually one terminus of the antiviral peptide, may be substituted with a lipophilic group, usually aliphatic or aralkyl, of from 8 to 36, usually 8 to 24 carbon atoms and fewer than two heteroatoms in the aliphatic chain, the heteroatoms usually being oxygen, nitrogen and sulfur. As further described below, the chain may be saturated or unsaturated, desirably having not more than 3 sites, usually not more than 2 sites of aliphatic unsaturation. Conveniently, commercially available aliphatic fatty acids, alcohols and amines may be used, such as caprylic acid, capric acid, lauric acid, myristic acid and myristyl alcohol, palmitic acid, palmitoleic acid, stearic acid and stearyl amine, oleic acid, linoleic acid, docosahexaenoic acid, etc. (see U.S. Pat. No. 6,225,444, hereby incorporated by reference). Preferred are unbranched, naturally occurring fatty acids between 14-22 carbon atoms in length. Other lipohilic molecules include glyceryl lipids and sterols, such as cholesterol. The lipophilic groups may be reacted with the appropriate functional group on the peptide in accordance with conventional methods, frequently during the synthesis on a support, depending on the site of attachment of the peptide to the support. Lipid attachment is useful where peptides may be introduced into the lumen of the liposome, along with other therapeutic agents (e.g. antiviral agents) for administering the peptides and agents into a host.

Increasing lipophilicity is also known to increase transport of compounds across endothelial cells and therefore useful in promoting uptake of such compounds from the intestine or blood stream into surrounding tissues.

The terminal amino group or carboxyl group of the antiviral peptide may be modified by alkylation, amidation, or acylation to provide esters, amides or substituted amino groups, where the alkyl or acyl group may be of from about 1 to 30, usually 1 to 24, preferably either 1 to 3 or 8 to 24, particularly 12 to 18 carbon atoms. The peptide or derivatives thereof may also be modified by acetylation or methylation to alter the chemical properties, for example lipophilicity. Other modifications include deamination of glutamyl and asparaginyl residues to the corresponding glutamyl and aspartyl residues, respectively; hydroxylation of proline and lysine; phosphorylation of hydroxyl groups of serine or threonine; and methylation of amino groups of lysine arginine, and histidine side chains. Depending upon their intended use, particularly for administration to mammalian hosts, the subject peptides may be modified or attached to other compounds for the purposes of incorporation into carrier molecules, changing peptide bioavailability, extend or shorten half-life, control distribution to various tissues or the blood stream, diminish or enhance binding to blood components, and the like. The subject peptides may be bound to these other components by linkers which are cleavable or non-cleavable in the physiological environment such as blood, cerebrospinal fluid, digestive fluids, etc.

The peptides may be joined at any point of the peptide where a functional group is present, such as hydroxyl, thiol, carboxyl, amino, or the like. Desirably, modification will be at either the N-terminus or the C-terminus. For these purposes, the subject peptides may be modified by covalently attaching polymers, such as polyethylene glycol, polypropylene glycol, carboxymethyl cellulose, dextran, polyvinyl alcohol, polyvinylpyrrolidine, polyproline, poly (divinyl-ether-co-maleic anhydride), poly (styrene-c-maleic anhydride), etc. Water soluble polymers, such a polyethylene glycol and polyvinylpyrrolidine are known to decrease clearance of attached compounds from the blood stream as compared to unmodified compounds. The modifications can also increase solubility in aqueous media and reduce aggregation of the peptides.

In another aspect, the peptide is preferably conjugated to small molecules for detection and isolation of the peptides, and to target or transport the antiviral peptide into specific cells, tissues, and organs. Small molecule conjugates include haptens, which are substances that do not initiate an immune response when introduced by themselves into an animal. Generally, haptens are small molecules of molecular weight less than about 2 kD, and more preferably less that about 1 kD. Haptens include small organic molecules (e.g., p-nitrophenol, digoxin, heroin, cocaine, morphine, mescaline, lysergic acid, tetrahydrocannabinol, cannabinol, steroids, pentamidine, biotin, etc.). Binding to the hapten, for example for purposes of detection or purification, are done with hapten specific antibodies or specific binding partners, such as avidin which binds biotin.

Small molecules that target the conjugate to specific cells or tissues may also be used. It is known that presence of a biotin-avidin complex increases uptake of such modified peptides across endothelial cells. Linkage of peptides to carbohydrate moieties, for example to a p-glycoside through a serine residue on the peptide to form a ss-O linked glycoside, enhances transport of the glycoside derivative via glucose transporters. Both of these types of modifications are encompassed within the scope of the present invention.

The antiviral peptides may have attached various label moieties such as radioactive labels and fluorescent labels for detection and tracing. Fluorescent labels include, but are not limited to, fluorescein, eosin, Alexa Fluor, Oregon Green, rhodamine Green, tetramethylrhodamine, rhodamine Red, Texas Red, coumarin and NBD fluorophores, the QSY 7, dabcyl and dabsyl chromophores, BIODIPY, Cy5, etc.

In one aspect, the peptides are joined to a wide variety of other peptides or proteins for a variety of purposes. The peptides may be linked to peptides or proteins to provide convenient functionalities for bonding, such as amino groups for amide or substituted amine formation, e.g., reductive amination; thiol groups for thioether or disulfide formation; carboxyl groups for amide formation; and the like. Of particular interest are peptides of at least 2, more usually 3, and not more than about 60 lysine groups, particularly polylysines of from about 4 to 20, usually 6 to 18 lysine units, referred to as multiple antigenic peptide system (MAPS), where the subject peptides are bonded to the lysine amino groups, generally at least about 20%, more usually at least about 50%, of available amino groups, to provide a multipeptide product. In this way, molecules having a plurality of the subject peptides are obtained where the orientation of the subject peptides is in the same direction; in effect one has a linking group to provide for tail to tail di- or oligomerization.

In another aspect, the peptides are conjugated to other peptides or proteins for targeting the antiviral peptide to cells and tissues, or adding additional functionalities to the peptides of SEQ ID No. 1.

For targeting, the protein or peptide used for conjugation will be selected based on the cell or tissue being targeted for therapy. The proteins may also compromise poly-amino acids including, but not limited to, polyarginine; and polylysine, polyaspartic acid, etc., which may be incorporated into other polymers, such as polyethylene glycol, for preparation of vesicles or particles containing the conjugated peptides.

In another aspect, the subject peptides may be expressed in conjunction with other peptides or proteins, so as to be a portion of the polypeptide chain, either internal, or at the N- or C-terminus to form chimeric proteins or fusion proteins. By “fusion polypeptide” or “fusion protein” or “chimeric protein” herein is meant a protein composed of a plurality of protein components that, while typically joined in the native state, are joined by the respective amino and carboxy termini through a peptide linkage to form a continuous polypeptide. Plurality in this context means at least two, and preferred embodiments generally utilize three to twelve components, although more may be used. It will be appreciated that the protein components can be joined directly or joined through a peptide linker/spacer as outlined below.

### Peptide Synthesis

The antiviral peptides of the present invention may be prepared in a number of ways. Chemical synthesis of peptides are well known in the art. Solid phase synthesis is commonly used and various commercial synthetic apparatuses are available, for example automated synthesizers by Applied Biosystems Inc., Foster City, Calif.; Beckman; etc. Solution phase synthetic methods may also be used, although it is less convenient. By using these standard techniques, naturally occurring amino acids may be substituted with unnatural amino acids, particularly D-stereoisomers, and also with amino acids with side chains having different lengths or functionalities. Functional groups for conjugating to small molecules, label moieties, peptides, or proteins, or for purposes of forming cyclized peptides may be introduced into the molecule during chemical synthesis. In addition, small molecules and label moieties may be attached during the synthetic process. Preferably, introduction of the functional groups and conjugation to other molecules minimally affects the structure and function of the subject peptide.

The N- and C-terminus may be derivatized using conventional chemical synthetic methods. The antiviral peptides of the invention may contain an acyl group, such as an acetyl group.

Methods for acylating, and specifically for acetylating the free amino group at the N-terminus are well known in the art. For the C-terminus, the carboxyl group may be modified by esterification with alcohols or amidated to form-CONH2, CONHR, or CONR, wherein each R is a hybroxycarbyl (1-6 carbons). Methods of esterification and amidation are done using well known techniques.

The antiviral peptides of the present invention may also be present in the form of a salt, generally in a salt form which is pharmaceutical acceptable. These include inorganic salts of sodium, potassium, lithium, ammonium, calcium, magnesium, iron, zinc, copper, manganese, and the like. Various organic salts of the peptide may also be made with, including, but not limited to, acetic acid, propionic acid, pyruvic acid, maleic acid, succinic acid, tartaric acid, citric acid, benzoic acid, cinnamic acid, salicylic acid, etc.

Synthesis of the antiviral peptides and derivatives thereof may also be carried out by using recombinant techniques. For recombinant production using state-of-the-art expression systems, one may prepare a nucleic acid sequence which encodes a single peptide or preferably a plurality of the subject peptides in tandem with an intervening amino acid or sequence, which allows for cleavage to the single peptide or head to tail dimers.

The subject peptide may also be made as part of a larger peptide, which can be isolated and the peptide obtained by proteolytic cleavage or chemical cleavage. The particular sequence and the manner of preparation will be determined by convenience, economics, purity required, and the like. To prepare these compositions, a gene encoding a particular peptide, protein, or fusion protein is joined to a DNA sequence encoding the antiviral peptides of the present invention to form a fusion nucleic acid, which is introduced into an expression vector. Expression of the fusion nucleic acid is under the control of a suitable promoter and other control sequences, as defined below, for expression in a particular host cell or organism.

There are a variety of techniques available for introducing nucleic acids into viable cells. By “introduced” into herein is meant that the nucleic acid enters the cells in a manner suitable for subsequent expression of the nucleic acid. Exemplary for introducing the nucleic acids in vitro include the use of liposomes, Lipofectin, electroporation, microinjection, cell fusion, DEAE dextran, calcium phosphate precipitation, and biolistic particle bombardment. Techniques for transfer in vivo include direct introduction of the nucleic acid, use of viral vectors, typically retroviral vectors, and liposome mediated transfection, such as viral coated liposome mediated transfection.

For conjugating various molecules to the peptides of the present invention, functional groups on the peptides and the other molecule are reacted in presence of an appropriate conjugating (e.g., crosslinking) agent. The type of conjugating or crosslinking agent used will depend on the functional groups, such as primary amines, sulfhydryls, carbonyls, carbohydrates and carboxylic acids being used. Agents may be fixatives and crosslinking agents, which may be homobifunctional, heterobifunctional, or trifunctional crosslinking agents (Pierce Endogen, Chicago, Ill.). Commonly used fixatives and crosslinking agents include formaldehyde, glutaraldehyde, 1,1-bis(diazoacetyl)-2-phenylethane, N-hydroxysuccinimide esters, dissuccimidyl esters, maleimides (e.g., bis-N-maleimido-1-8-octane), and carbodiimides (e.g., N-ethyl-N′-(3-dimethylaminopropyl)-carbodiimide dicyclohexylcarbodiimide. Spacer molecules comprising alkyl or substituted alkyl chains with lengths of 2-20 carbons may be used to separate conjugates. Preferably, reactive functional groups on the peptide not selected for modification are protected prior to coupling of the peptide to other reactive molecules to limit undesired side reactions. By “protecting group” as used herein is a molecule bound to a specific functional group which is selectively removable to reexpose the functional group (see Greene, T. W. and Wuts, P. G. M. Protective Groups in Organic Synthesis (3rd ed.), John Wiley & Sons, Inc., New York, 1999). The peptides may be synthesized with protected amino acid precursors or reacted with protecting groups following synthesis but before reacting with crosslinking agent. Conjugations may also be indirect, for example by attaching a biotin moiety, which can be contacted with a compound or molecule which is coupled to streptavidin or avidin.

For peptides that have reduced activity in the conjugated form, the linkage between the peptides and the conjugated compound is chosen to be sufficiently labile to result in cleavage under desired conditions, for example after transport to desired cells or tissues. Biologically labile covalent bonds, e.g., imimo bonds and esters, are well known in the art (see U.S. Pat. No. 5,108,921, hereby incorporated by reference). These modifications permit administration of the peptides in potentially a less active form, which is then activated by cleavage of the labile bond.

In a preferred embodiment, the antiviral peptides of the present invention may be purified or isolated after synthesis or expression. By “purified” or “isolated” is meant free from the environment in which the peptide is synthesized or expressed and in a form where it can be practically used. Thus purified or isolated is meant that the peptide or its derivative is substantially pure, i.e., more than 90% pure, preferably more than 95% pure, and preferably more than 99% pure. The peptides and derivatives thereof may be purified and isolated by way known to those skilled in the art, depending on other components present in the sample. Standard purification methods include electrophoretic, immunological, and chromatographic techniques, including ion exchange, hydrophobic, affinity, size exclusion, reverse phase HPLC, and chromatofocusing. The proteins may also be purified by selective solubility, for instance in the presence of salts or organic solvents. The degree of purification necessary will vary depending on use of the subject peptides. Thus, in some instances no purification will be necessary.

For the most part, the compositions used will comprise at least 20% by weight of the desired product, more usually at least about 75% by weight, preferably at least about 95% by weight, and usually at least about 99.5% by weight, relative to contaminants related to the method of product preparation, the purification procedure, and its intended use, for example with a pharmaceutical carrier for the purposes of therapeutic treatment. Usually, the percentages will be based upon total protein.

### Pharmaceutical Formulations

The subject compositions, either alone or in combination, may be used in vitro, ex vivo, and in vivo depending on the particular application. In accordance, the present invention provides for administering a pharmaceutical composition comprising a pharmaceutically acceptable carrier and a pharmacologically effective amount of one or more of the subject peptides, or suitable salts thereof.

The pharmaceutical composition may be formulated as powders, granules, solutions, suspensions, aerosols, solids, pills, tablets, capsules, gels, topical cremes, suppositories, transdermal patches, etc.

As indicated above, pharmaceutical acceptable salts of the peptides is intended to include any art recognized pharmaceutically acceptable salts including organic and inorganic acids and/or bases. Examples of salts include sodium, potassium, lithium, ammonium, calcium, as well as primary, secondary, and tertiary amines, esters of lower hydrocarbons, such as methyl, ethyl, and propyl. Other salts include organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” comprises any of standard pharmaceutical accepted carriers known to those of ordinary skill in the art in formulating pharmaceutical compositions. Thus, the peptides of SEQ ID No. 1, by themselves, such as being present as pharmaceutical acceptable salts, or as conjugates, or nucleic acid vehicles encoding such peptides, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients. The pharmaceutical compositions also contain anti-retroviral agents when such agents are part of the compositions. Additionally, the formulations may include bactericidal agents, stabilizers, buffers, emulsifiers, preservatives, sweetening agents, lubricants, or the like. If administration is by oral route, the peptides of SEQ ID No. 1 may be protected from degradation by using a suitable enteric coating, or by other suitable protective means, for example internment in a polymer matrix such as microparticles or pH sensitive hydrogels.

Suitable formulations may be found in, among others, Remington's Pharmaceutical Sciences, 17th edition, Mack Publishing Co., Philadelphia, Pa., 1985 and Handbook of Pharmaceutical Excipients, 3rd Ed, Kibbe, A. H. ed., Washington D.C., American Pharmaceutical Association, 2000; hereby incorporated by reference in their entirety. The pharmaceutical compositions described herein can be made in a manner well known to those skilled in the art (e.g., by means conventional in the art, including mixing, dissolving, granulating, levigating, emulsifying, encapsulating, entrapping or lyophilizing processes).

Additionally, the peptides of SEQ ID No. 1, either alone or with the anti-retroviral agents may also be introduced or encapsulated into the lumen of liposomes for delivery and for extending life time of the peptide formulations ex vivo or in vivo. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids; and other lipids.

Cationic lipids are also suitable for forming liposomes. Generally, the cationic lipids have an net positive charge and have a lipophilic portion, such as a sterol or an acyl or diacyl side chain.

Preferably, the head group is positively charged. Typical cationic lipids include 1, 2-dioleyloxy-3-(trimethylamino) propane; N-[1-(2,3,-ditetradecycloxy) propyl]-N, N-dimethyl-N-N-hydroxyethylammonium bromide; N-[1-(2,3-dioleyloxy) propyl]-N,N-dimethyl-N-hydroxy ethylammonium bromide; N-[1-(2,3-dioleyloxy) propyl]-N,N,N-trimethylammonium chloride; 3-[N—(N′,N′-dimethyl aminoethane) carbamoyl] cholesterol and dimethyldioctadecylammonium.

Of particular interest are fusogenic liposomes, which are characterized by their ability to fuse with a cell membrane upon appropriate change in physiological condition or by presence of fusogenic component, particularly a fusogenic peptide or protein. In one aspect, the fusogenic liposomes are pH and temperature sensitive in that fusion with a cell membrane is affected by change in temperature and/or pH. Generally, pH sensitive liposomes are acid sensitive. Thus, fusion is enhanced in physiological environments where the pH is mildly acidic, for example the environment of a lysosome, endosome and inflammatory tissues. This property allows direct release of the liposome contents into the intracellular environment following endocytosis of liposomes.

Another form of fusogenic liposomes comprise liposomes that contain a fusion enhancing agent. That is, when incorporated into the liposome or attached to the lipids, the agents enhance fusion of the liposome with other cellular membranes, thus resulting in delivery of the liposome contents into the cell. The agents may be fusion enhancing peptides or proteins, including hemaggulutinin HA2 of influenza virus; Sendai virus envelope glycoproteins; vesicular stomatitis virus envelope glycoproteins (VSV-G) glycoprotein; peptide segments or mimics of fusion enhancing proteins; and synthetic fusion enhancing peptides.

Liposomes also include vesicles derivatized with a hydrophilic polymer to extend the circulation lifetime in vivo. Hydrophilic polymers for coating or derivation of the liposomes include polyethylene glycol, polyvinylpyrrolidone, polyvinylmethyl ether, polyaspartamide, hydroxymethyl cellulose, hydroxyethyl cellulose, and the like. In addition, as described above, attaching proteins that bind a cell surface protein which is endocytosed, e.g., capsid proteins or fragments thereof trophic for a particular cell types and antibodies for cell surface proteins which undergo internalization may be used for targeting and/or facilitating uptake of the liposomes to specific cells or tissues.

Liposomes are prepared by ways well known in the art. One typical method is the lipid film hydration technique in which lipid components are mixed in an organic solvent followed by evaporation of the solvent to generate a lipid film. Hydration of the film in aqueous buffer solution, preferably containing the subject peptide or nucleic acid, results in an emulsion, which is sonicated or extruded to reduce the size and polydispersity. Other methods include reverse-phase evaporation, freezing and thawing of phospholipid mixtures, and ether infusion.

In another preferred embodiment, the carriers are in the form of microparticles, microcapsules, micropheres and nanoparticles, which may be biodegradable or non-biodegradable (see for example, Microencapsulates: Methods and Industrial Applications, Drugs and Pharmaceutical Sciences, Vol 73, Benita, S. ed, Marcel Dekker Inc., New York, 1996; incorporated by reference). As used herein, microparticles, microspheres, microcapsules and nanoparticles mean a particle, which is typically a solid, containing the substance to be delivered. The substance is within the core of the particle or attached to the particle's polymer network. Generally, the difference between microparticles (or microcapsules or microspheres) and nanoparticles is one of size. As used herein, microparticles have a particle size range of about 1 to about >1000 microns. Nanoparticles have a particle size range of about 10 to about 1000 nm.

A variety of materials are useful for making microparticles. Non-biodegradable microcapsules and microparticles include, but not limited to, those made of polysulfones, poly (acrylonitrile-co-vinyl chloride), ethylene-vinyl acetate, hydroxyethylmethacrylate-methyl-methacrylate copolymers. These are useful for implantation purposes where the encapsulated peptide diffuses out from the capsules.

In another aspect, the microcapsules and microparticles are based on biodegradable polymers, preferably those that display low toxicity and are well tolerated by the immune system. These include protein based microcapsulates and microparticles made from fibrin, casein, serum albumin, collagen, gelatin, lecithin, chitosan, alginate or poly-amino acids such as poly-lysine. Biodegradable synthetic polymers for encapsulating may comprise polymers such as polylactide (PLA), polyglycolide (PGA), poly (lactide-co-glycolide) (PLGA), poly (caprolactone), polydioxanone trimethylene carbonate, polyhybroxyalkonates (e.g., poly (ss-hydroxybutyrate)), poly (y-ethyl glutamate), poly (DTH iminocarbony (bisphenol A iminocarbonate), poly (ortho ester), and polycyanoacrylate. Various methods for making microparticles containing the subject compositions are well known in the art, including solvent removal process, emulsification and evaporation, spray drying, and extrusion methods.

Another type of carrier is nanoparticles, which are generally suitable for intravenous administrations. Submicron and nanoparticles are generally made from amphiphilic diblock, triblock, or multiblock copolymers as is known in the art. Polymers useful in forming nanoparticles include, but are limited to, poly (lactic acid), poly (lactide-coglycolide), blends of poly (lactide-co-glycolide) and polycarprolactone, diblock polymer poly (l-leucine-block-l-glutamate), diblock and triblock poly (lactic acid) (PLA) and poly (ethylene oxide) (PEO), acrylates, arylamides, polystyrene, and the like. As described for microparticles, nanoparticles may be non-biodegradable or biodegradable.

Nanoparticles may be also be made from poly (alkylcyanoacrylate), for example poly (butylcyanoacrylate), in which the peptide of SEQ ID No. 1 is absorbed onto the nanoparticles and coated with surfactants (e.g., polysorbate 80). Methods for making nanoparticles are similar to those for making microparticles and include, among others, emulsion polymerization in continuous aqueous phase, emulsification-evaporation, solvent displacement, and emulsification-diffusion techniques (see Kreuter, J. Nano-particle Preparation and Applications, In Microcapsules and nanoparticles in medicine and pharmacy,” (M. Donbrow, ed.), pg. 125-148, CRC Press, Boca Rotan, Fla., 1991; incorporated by reference). Hydrogels are also useful in delivering the subject agents into a host. Generally, hydrogels are crosslinked, hydrophilic polymer networks permeable to a wide variety of drug compounds, including peptides. Hydrogels have the advantage of selective trigger of polymer swelling, which results in controlled release of the entrapped drug compound. Depending on the composition of the polymer network, swelling and subsequent release may be triggered by a variety of stimuli, including pH, ionic strength, thermal, electrical, ultrasound, and enzyme activities. Non-limiting examples of polymers useful in hydrogel compositions include, among others, those formed from polymers of poly (lactideco-glycolide), poly (N-isopropylacrylamide); poly (methacrylic acid-g-polyethylene glycol); polyacrylic acid and poly (oxypropylene-co-oxyethylene) glycol; and natural compounds such as chrondroitin sulfate, chitosan, gelatin, or mixtures of synthetic and natural polymers, for example chitosanpoly (ethylene oxide). The polymers are crosslinked reversibly or irreversibly to form gels embedded with the peptides of the present invention.

In one preferred embodiment, the gel polymers are acrylic acid polymers, preferably carbomers (e.g., carboxypolymethylene), such as Carbopol (e.g., Carbopol 420-430, 475, 488, 493, 910, 934P, 974P, and the like; Brock et al., Pharmacotherapy 14: 430-437 (1994)), which are non-linear polymers of acrylic acid crosslinked with polyalkenyl polyether. Others types of carbomers include acrylic acids crosslinked with polyfunctional compounds, such as polyallysucrose. In addition to the advantage of hydrating and swelling to a gel, which entraps the subject compounds and limits their release, carbomer gels are mucoadhesive. The polymers adheres to the intestinal mucosal membrane, thus resulting in local delivery of the peptides, hereby incorporated by reference). In addition, these polymers have the added advantage of limiting intestinal protease activity.

The concentrations of the peptides of SEQ ID No. 1 or nucleic acid encoding therefore and the anti-retroviral agents will be determined empirical in accordance with conventional procedures for the particular purpose.

Generally, for administering the peptides and anti-retroviral agents ex vivo or in vivo for therapeutic purposes, the subject formulations are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms of the disorder or disease.

The amount administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but is not limited to, size and weight of the subject, the age and sex of the subject, the severity of the symptom, the stage of the disease, method of delivery of the agent, half-life of the agents, and efficacy of the agents. Stage of the disease to consider include whether the disease is acute or chronic, relapsing or remitting phase, and the progressiveness of the disease. Determining the dosages and times of administration for a therapeutically effective amount are well within the skill of the ordinary person in the art.

For any compounds used in the present invention, therapeutically effective dose is readily determined by methods well known in the art. For example, an initial effective dose can be estimated initially from cell culture assays. An indicator of HIV infection and/or inflammatory response or may be used, such as viral replication, presence of viral expression products, expression levels of pro-inflammatory cytokines, or inhibition of CTL activity. A dose can then be formulated in animal models to generate a circulating concentration or tissue concentration, including that of the ICso (i.e., dose lethal to about 50% of cells in the cell culture) as determined by the cell culture assays.

In addition, the toxicity and therapeutic efficacy are generally determined by cell culture assays and/or experimental animals, typically by determining a LD50 (lethal dose to 50% of the test population) and ED50 (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the detailed disclosure provided herein.

Generally, in the case where a formulations are administered directly to a host, the present invention provides for a bolus or infusion of the subject composition that will administered in the range of about 0.1-100, more usually from about 1-25 mg/kg body weight of host. The amount will generally be adjusted depending upon the half-life of the peptide and anti-retroviral agent, where the half life will generally be at least one minute, more usually at least about 10 min, desirably in the range of about 10 min to 12 h. Short half-lives are acceptable, so long as efficacy can be achieved with individual dosages, continuous infusion, or repetitive dosages. Formulations for administration may be presented in unit a dosage form, e.g., in ampules, capsules, pills, or in multidose containers or injectables.

Dosages in the lower portion of the range and even lower dosages may be employed, where the peptide has an enhanced half-life or is provided as a depot, such as a slow release composition comprising particles, a polymer matrix which maintains the peptide over an extended period of time (e.g., a collagen matrix, carbomer, etc.), use of a pump which continuously infuses the peptide over an extended period of time with a substantially continuous rate, or the like. The host or subject may be any mammal including domestic animals, pets, laboratory animals, primates, particularly humans subjects.

In addition to administering the subject peptide compositions directly to a cell culture in vitro, to particular cells ex vivo, or to a mammalian host in vivo, nucleic acid molecules (DNA or RNA) encoding the subject peptides may also be administered thereto, thereby providing an effective source of the subject peptides for the application desired. As described above, nucleic acid molecules encoding the subject peptides may be cloned into any of a number of well known expression plasmids and/or viral vectors, preferably adenoviral or retroviral vectors under the transcriptional regulation of control sequences which function to promote expression of the nucleic acid in the appropriate environment. Such nucleic acid-based vehicles may be administered directly to the cells or tissues ex vivo (e.g., ex vivo viral infection of cells for transplant of peptide producing cells) or to a desired site in vivo, e.g. by injection, catheter, orally (e.g., hydrogels), and the like, or, in the case of viral-based vectors, by systemic administration. Tissue specific promoters may optionally be employed, assuring that the peptide of interest is expressed only in a particular tissue or cell type of choice. Methods for recombinantly preparing such nucleic acid-based vehicles are well known in the art, as are techniques for administering nucleic acid-based vehicles for peptide production.

For the purposes of this invention, the methods of administration is chosen depending on the condition being treated, the form of the subject compositions, and the pharmaceutical composition.

Administration of the peptides of SEQ ID No. 1 and anti-retroviral agents can be done in a variety of ways, including, but not limited to, cutaneously, subcutaneously, intravenously, orally, topically, transdermally, intraperitoneally, intramuscularly, nasally, and rectally (e.g., colonic administration).

For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, oral administration can be accompanied by rectal or topical administration to the affected area. Alternatively, oral administration is used in conjunction with intravenous or parenteral injections.

In one preferred embodiment, the method of administration is by oral delivery, in the form of a powder, tablet, pill, or capsule. Pharmaceutical formulations for oral administration may be made by combining one or more peptide and anti-retroviral agent with suitable excipients, such as sugars (e.g., lactose, sucrose, mannitol, or sorbitol), cellulose (e.g., starch, methyl cellulose, hydroxylmethyl cellulose, carbonxymethyl cellulose, etc.), gelatin, glycine, saccharin, magnesium carbonate, calcium carbonate, polymers such as polyethylene glycol or polyvinylpyrrolidone, and the like. The pills, tablets, or capsules may have an enteric coating, which remains intact in the stomach but dissolves in the intestine. Various enteric coating are known in the art, a number of which are commercially available, including, but not limited to, methacrylic acid-methacrylic acid ester copolymers, polymer cellulose ether, cellulose acetate phathalate, polyvinyl acetate phthalate, hydroxypropyl methyl cellulose phthalate, and the like. Alternatively, oral formulations of the peptides of SEQ ID No. 1 are in prepared in a suitable diluent. Suitable diluents include various liquid form (e.g., syrups, slurries, suspensions, etc.) in aqueous diluents such as water, saline, phosphate buffered saline, aqueous ethanol, solutions of sugars (e.g. sucrose, mannitol, or sorbitol), glycerol, aqueous suspensions of gelatin, methyl cellulose, hydroxylmethyl cellulose, cyclodextrins, and the like. As used herein, diluent or aqueous solutions also include infant formula, given that various forms of colitis can affect infants and children.

In some embodiments, lipohilic solvents are used, including oils, for instance vegetable oils, peanut oil, sesame oil, olive oil, corn oil, safflower oil, soybean oil, etc.); fatty acid esters, such as oleates, triglycerides, etc.; cholesterol derivatives, including cholesterol oleate, cholesterol linoleate, cholesterol myristilate, etc.; liposomes; and the like.

In another preferred embodiment, administration is done rectally. This may use formulations suitable for topical application in the form of salves, tinctures, cremes, or for application into the lumen of the intestine by use of compositions in the form of suppositories, enemas, foams, etc. Suppositories may contain conventional suppository bases such as cocoa butter, carbowaxes, polyethylene glycols, or glycerides, which are solid or semi-solid at room temperature but liquid at body temperature.

In yet another preferred embodiment, the administration is carried out cutaneously, subcutaneously, intraperitonealy, intramuscularly and intravenously. As discussed above, these are in the form of antiviral peptides dissolved or suspended in suitable aqueous medium, as discussed above. Additionally, the pharmaceutical compositions for injection may be prepared in lipophilic solvents, which include, but is not limited to, oils, such as vegetable oils, olive oil, peanut oil, palm oil soybean oil, safflower oil, etc; synthetic fatty acid esters, such as ethyl oleate or triglycerides; cholesterol derivatives, including cholesterol oleate, cholesterol linoleate, cholesterol myristilate, etc.; or liposomes, as described above. The compositions may be prepared directly in the lipophilic solvent or preferably, as oil/water emulsions.

The delivery systems also include sustained release or long term delivery methods, which are well known to those skilled in the art. By “sustained release or” “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the subject peptide, such as biodegradable polymers described above; pumps, including peristaltic pumps and fluorocarbon propellant pumps; osmotic and mini-osmotic pumps; and the like.

Peristaltic pumps deliver a set amount of drug with each activation of the pump, and the reservoir can be refilled, preferably percutaneously through a port. A controller sets the dosage and can also provides a readout on dosage delivered, dosage remaining, and frequency of delivery. Fluorocarbon propellant pumps utilize a fluorocarbon liquid to operate the pump. The fluorocarbon liquid exerts a vapor pressure above atmospheric pressure and compresses a chamber containing the drug to release the drug. Osmotic pumps (and mini-osmotic pumps) utilize osmotic pressure to release the drug at a constant rate. The drug is contained in an impermeable diaphragm, which is surrounded by the osmotic agent. A semipermeable membrane contains the osmotic agent, and the entire pump is housed in a casing. Diffusion of water through the semipermeable membrane squeezes the diaphragm holding the drug, forcing the drug into bloodstream, organ, or tissue. These and other such implants are particularly useful in treating a inflammatory disease condition, especially those manifesting recurring episodes or which are progressive in nature, by delivering the peptides of the invention via systemic (e.g., intravenous or subcutaneous) or localized doses in a sustained, long term manner.

The present invention also encompasses the therapeutic combinations disclosed herein in the form of a kit or packaged formulation. A kit or packaged formulation as used herein includes one or more dosages of an immunomodulating peptide, and salts thereof, and at least one anti-retroviral agent, in a container holding the dosages together with instructions for simultaneous or sequential administration to an HIV-infected patient. For example, the package may contain the peptides along with a pharmaceutical carrier combined in the form of a powder for mixing in an aqueous solution, which can be ingested by the afflicted subject. Another example of packaged drug is a reloaded pressure syringe, so that the compositions may be delivered colonically. The package or kit includes appropriate instructions, which encompasses diagrams, recordings (e.g., audio, video, compact disc), and computer programs providing directions for use of the combination therapy.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching.

### EXAMPLES

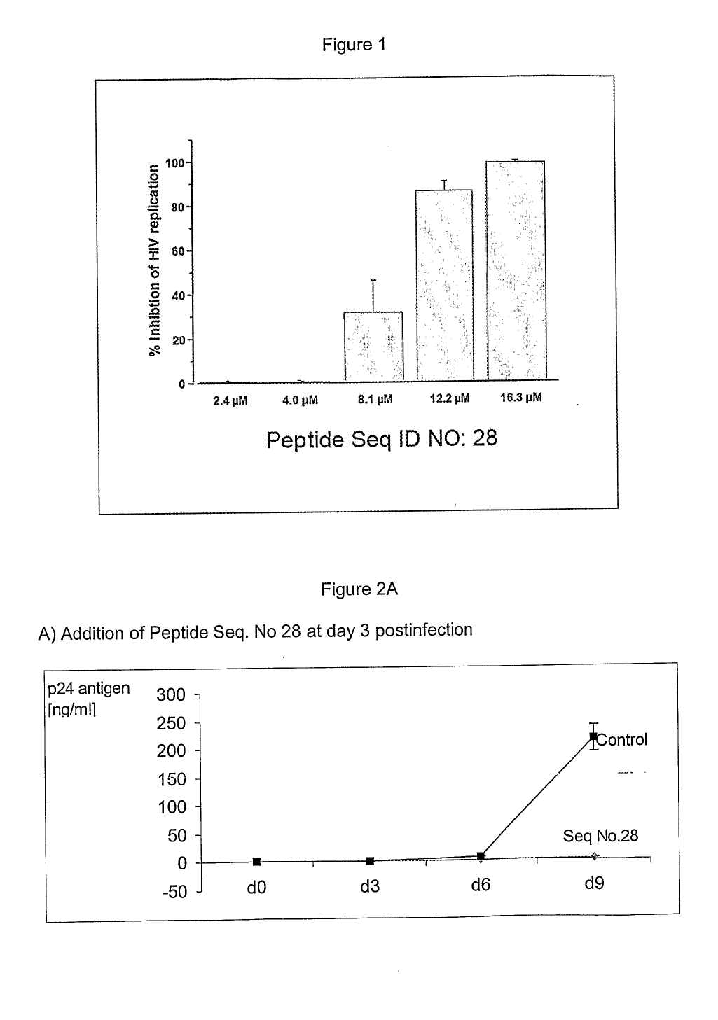

FIG. 1: Inhibition by Peptide Seq ID NO: 28 of HIV replication in a concentration-dependent manner. PM1 cells were de novo infected with macrophage-trophic HIV-1_{Ba-L} strain in the presence of Peptide Seq ID NO: 28. HIV replication was monitored by p24 assay as described below. Peptide Seq ID NO: 28 inhibits HIV replication in a concentration-dependent manner with IC₅₀ (half-maximal inhibitory constant) of 10 μM. This indicates that the Peptide Seq ID NO: 28 specifically blocks HIV replication.

FIG. 2: Antiviral activity of Peptide Seq ID NO: 28 on HIV postinfected cells. PM1 cells were infected with HIV-1Ba-L for 3 hours and subsequently cultivated with RPMI. At day 3 (A) or day 6 (B) postinfection cells were split and either DMSO (control) or Peptide Seq No. 28 (20 μg/ml) were added. HIV replication was monitored by p24 assay as described below during further incubation for 9 days (0d-9d) at day: 0d, 3d, 6d and 9d. Peptide Seq ID NO: 28 is able to block HIV replication in already infected cells.

FIG. 3: Inhibition by Peptide Seq ID NO: 28 of replication of a T-trophic HIV-1_{NL4-3} strain. Jurkat cells were infected with the T-cell trophic strain HIV-1 NL4-3 in the presence of either 10 μg/ml (8.1 μM) or 20 μg/ml (16.3 μM) Peptide Seq ID NO: 28. HIV replication was monitored by p24 assay as described below at day 6 (d6) and day 9 (d9) postinfection. Peptide Seq ID NO: 28 effectively inhibits HIV replication at both concentrations. This indicates that Peptide Seq ID NO: 28 can block replication of both T-trophic and M-trophic HIV.

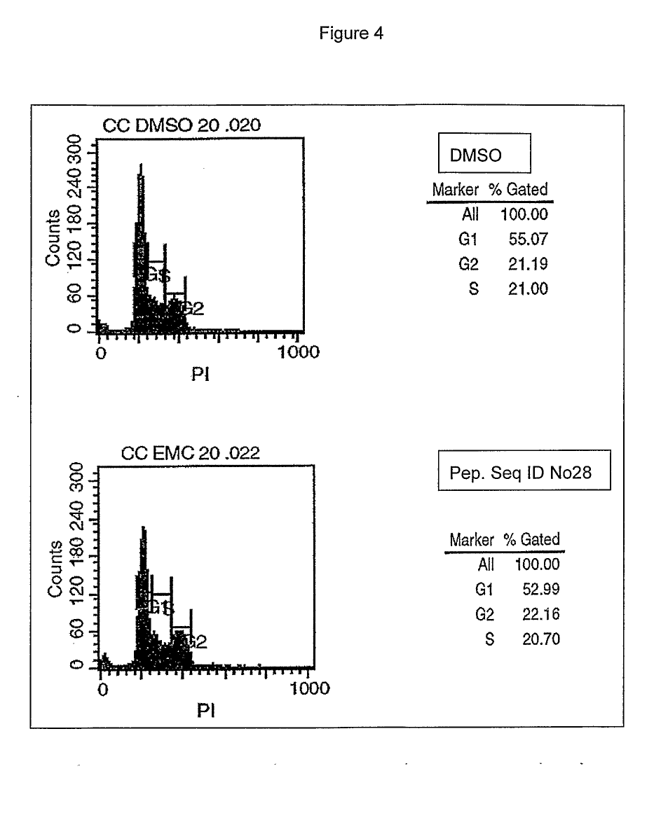

FIG. 4: Cell cycle-analysis of PM1 cells in the presence of antiviral peptide Seq ID NO: 28 or DMSO (control). Distribution of cells in G1, G2, M and S-Phase was similar when treated with either Peptide Seq ID NO: 28 or DMSO for 21 days. This indicates that the antiviral peptides do not induce cell cycle arrest and consequently do not inhibit cell proliferation.

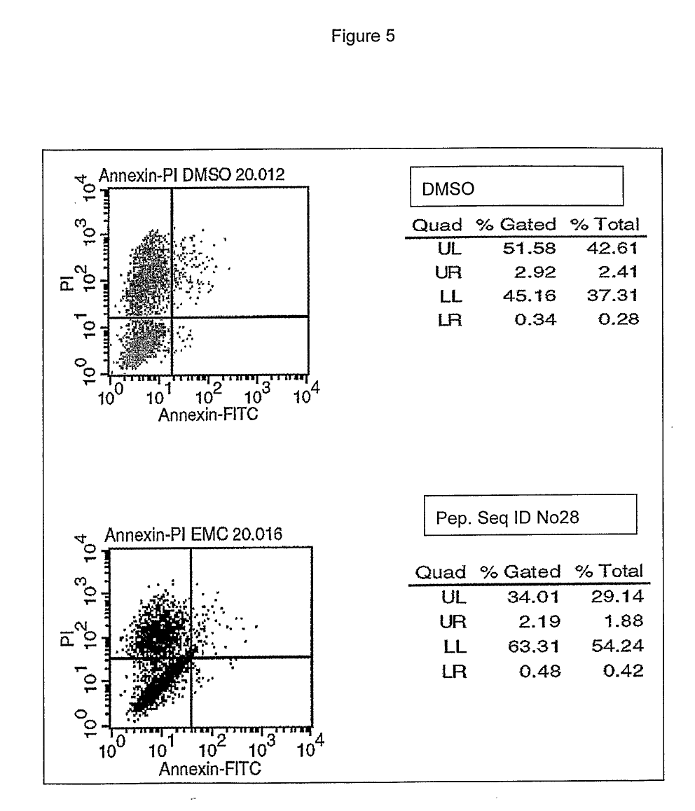

FIG. 5: Apoptosis of PM1 cells in the presence of antiviral peptide Seq ID NO: 28 or DMSO (control). FACS analysis pattern of both peptide Seq ID NO: 28 and DMSO treated cells did not show any difference indication that the inventive peptides do not induce apoptosis.

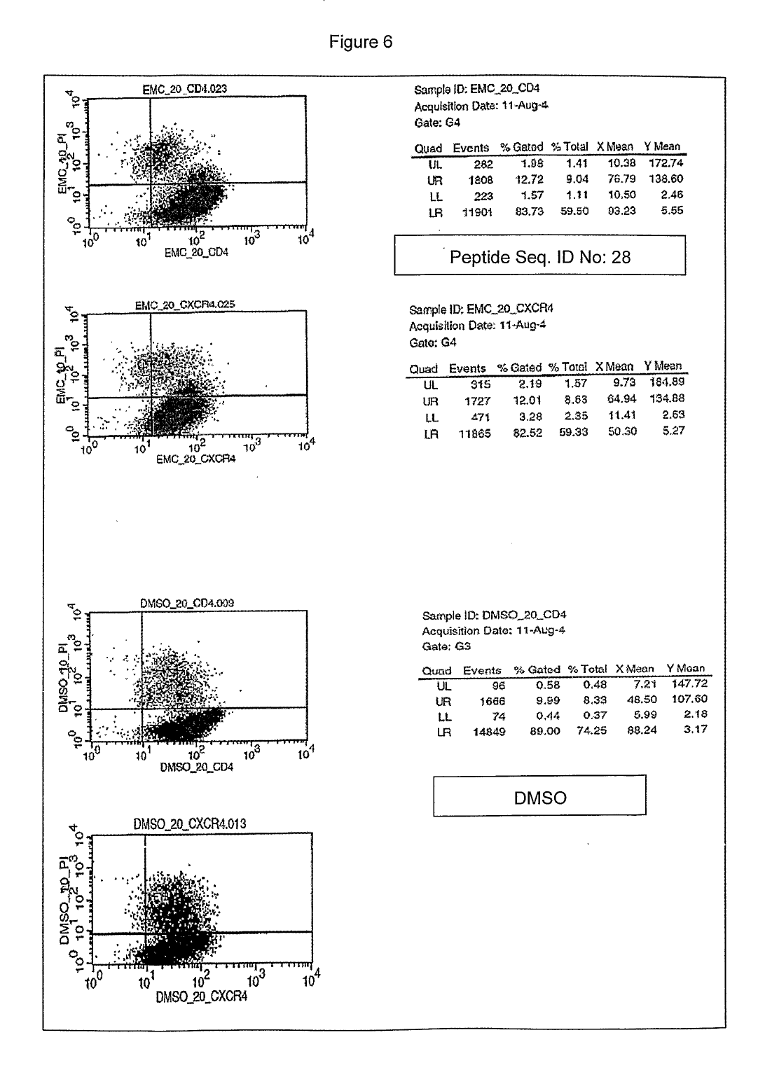

FIG. 6: Expression of CD4 and CCR5 receptors in the presence of antiviral peptide Seq ID NO: 28 or DMSO (control). Cells were assayed by FACS (FACSCalibur, Becton Dickinson) using a commercially available FITC-coupled antibody against CD4 and CCR5 receptor according to the manufacturer's protocol (Bender Medsystems # BMS306FI). Equal level of both CD4 and CCR5 were found on PM1 cells when treated with either peptide Seq ID NO: 28 or DMSO. This shows that peptide Seq ID NO: 28 does not downregulate the cellular docking receptors for HIV to inhibit the HIV cell entry.

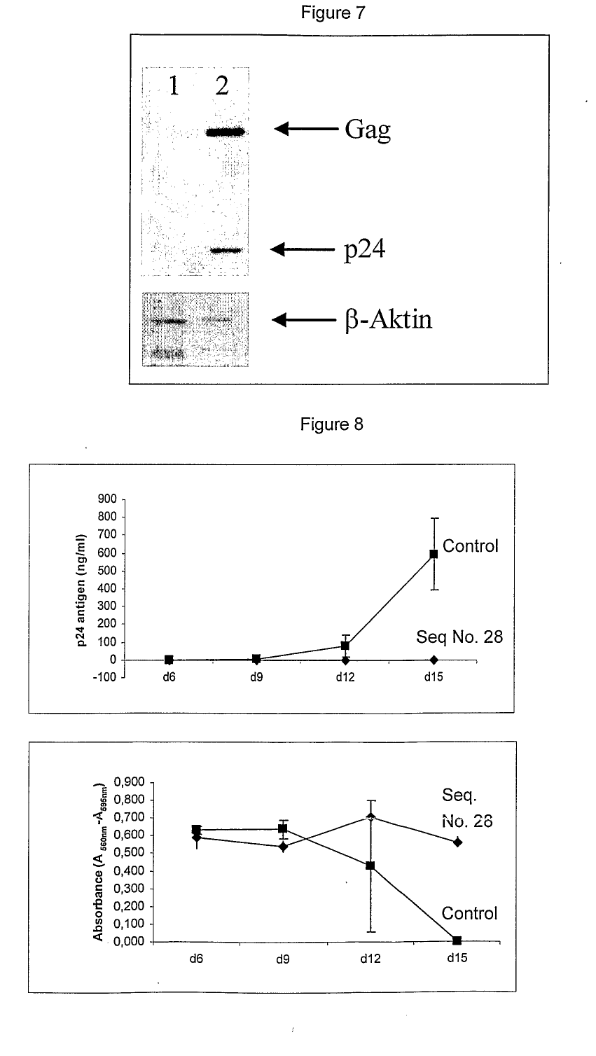

FIG. 7: Protein expression of p24 and Gag in PM1 cells infected with HIV-1 BaL for 9 days in the presence of antiviral peptide Seq ID NO: 28 (lane 1) or DMSO (control, lane 2). Cells were lysed and proteins were analysed with SDS-PAGE and Western Blotting using an anti-p24 antibody. In the presence or peptide Seq ID NO: 28 no protein expression of both p24 and Gag was detected in contrast to the DMSO treated cells indicating that peptide Seq ID NO: 28 blocks specifically protein translation of viral proteins.

FIG. 8: Inhibition by Peptide Seq ID NO: 28 of replication of the omni-resistant HIV stain FE9. PM1 cells were incubated with HIV-1 FE9 for 2 hours and subsequently HIV replication was monitored by p24 assay as described below at day d6, d9, d12 and d15. In control (DMSO) treated cells HIV-1 FE9 replication could be detected at day 12 postinfection. Cell viability as monitored by Trypan-blue staining and photometric analysis was reduced in control treated cells at day 12 reflecting the cell death by HIV.

At day 15 no viable cell was detected when treated with DMSO and consequently high amount of HIV were determined. In contrast, when treated with peptide Seq ID NO: 28 al cells were viable at day 15 and no virus was determined. In summary, peptide Seq ID NO: 28 is a potent antiviral peptide effectively blocking replication of multi-resistant HIV strains without any cell toxicity.

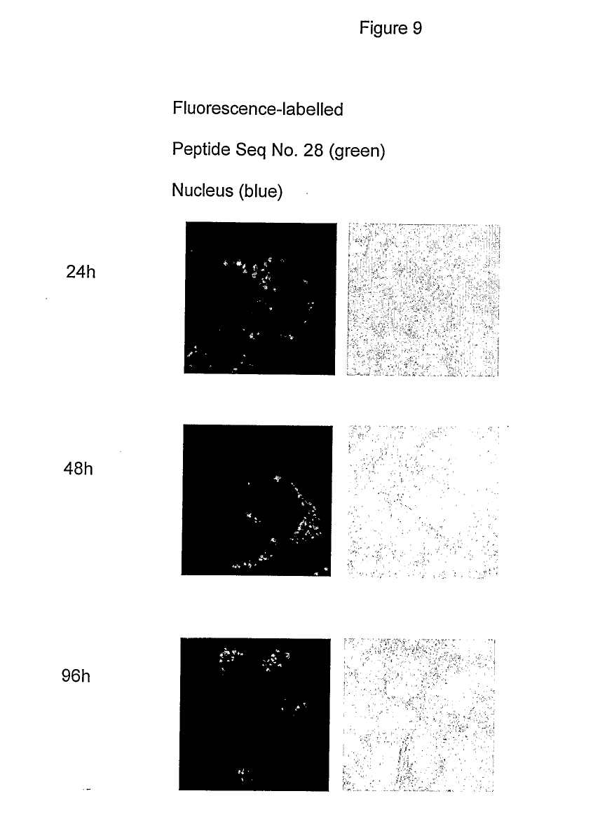

FIG. 9: Specific localization of Peptide Seq ID NO: 28 within the cell. HeLaCD4-CAT cells were incubated with Peptide Seq. No. 28 coupled to carboxyfluorescein (2 μg/ml) for 24, 48 and 96 hours. Cell nuclei were stained with DRAQ5 (1:1000). Peptide Seq ID NO: 28 can be specifically localized within organelles close to the nucleus. Distribution of peptide Seq ID NO: 28 does not change during 96 hours. Presumably, peptide Seq ID NO: 28 binds to a cellular protein localized close to the nucleus which is required for HIV replication and prevents expression of viral proteins.

### MATERIALS AND METHODS

### HIV Detection

HIV replication was measured either by p24 HIV Antigen Capture Assay Kit (ELISA; HIVAG-1 Monoclonal kit B1A011; Abbott) following the manufacturer's recommendation, or by using the Quantiplex HIV-1 RNA 3.0 Assay (bDNA) system (Chiron Diagnostics). The latter assay is a signal amplification nucleic acid probe assay (employing a sandwich nucleic acid hybridization procedure) for direct quantitation of HIV RNA in human plasma. There, in brief, HIV-1 is first concentrated from plasma by centrifugation. After release of genomic HIV-1 RNA from the virions, the RNA is captured to a microwell by a set of specific, synthetic oligonucleotide capture probes. A set of target probes hybridize to both the viral RNA and the pre-amplifier probes. The capture probes, comprised of 17 individual capture extenders, and the target probes, comprised of 81 individual target extenders, bind to different regions of the pol gene of the viral RNA. The amplifier probe hybridizes to the pre-amplifier forming a branched DNA (bDNA) complex. Then, multiple copies of an alkaline phosphatase labeled probe are hybridized to this immobilized bDNA complex. Detection is achieved by incubating the entire complex with a chemiluminescent substrate. Light emission is directly proportional to the amount of HIV-1 RNA present in each sample, and results are recorded as relative light units by an appropriate analyzer.

### Cells and HIV Strains

Jurkat cells (species: human T-cell leukemia; special characteristics: cells are permissive for growth of T-cell trophic HIV strains; source: ATCC Cat. No. TIB 152) or PM1 cells (species: clonal derivative of human HUT 78 cells; special characteristics: cells are permissive for growth of macrophage-trophic and T-cell trophic HIV strains; source: Dr. Marvin Reitz, courtesy of the NIH AIDS Research and Reference Reagent Program Cat. No. 3038) were stimulated with Phytohemagglutinin (PHA-P, Product No. L9132 from Sigma) in a concentration of 2 μg/ml (1.5×10⁶ cells/ml) and Hexadimethrine Bromide (Polybrene, Product No. H9268 from Sigma) in a concentration of 2 μg/ml (1.5×10⁶ cells/ml) in RPMI 1640 medium containing 10% fetal calf serum (FCS, Pansystems GmbH) and antibiotics for 3 days.

### Inhibition of T-cell-trophic and Macrophage-Trophic HIV-1

For HIV-1 infection, 5×10⁷ cells were resuspended in 500 μl culture medium without test drugs and incubated in a 50 ml blue-cap-tube at 37° C. for 5 hrs with HIV-1 high titer viral stocks. Jurkat cells were infected with the T-cell trophic strain HIV-1 NL4-3, PM1 cells with the macrophage trophic strain HIV-1 Ba-L (both from the NIH AIDS Research and Reference Reagent Program). After infection, cells were washed twice with PBS without Ca²⁺ and Mg²⁺ to avoid false positive p24 antigen determination. Cells were resuspended, and identical aliquots (1×10⁶/ml) of infected cells were further cultured in 10 ml medium with test drugs at various concentrations, or in medium with DMSO as control for calculation of the inhibition of virus replication [in %].

Culture medium was changed and cells were split at days 3 and 7 of the experiments. Viability of the cells (Trypan-blue staining), cell counts and p24 antigen levels were determined at days 3, 6, 9, 12 and 15 of the experiments. No significant differences in cell viability were observed (˜85-90% living cells) in the presence of Peptide SEQ ID NO: 28. In the presence of DMSO significant reduction in cell viability was observed at day 15 (50% living cells) due to viral induced cell death.

Complete inhibition of virus replication (100%) was observed after 3, 6, 9, 12 and 12 days treatment with Peptide SEQ ID NO:28 (see table below).

PM1 cells de novo infected with macrophage-trophic HIV-1_{Ba-L} strain

| | |
|---|---|
| DMSO (solvent | Peptide SEQ ID NO: 28 |
| control) | 16.3 μM |

| | | | |
|---|---|---|---|
| d3: | 0% | d3: | 100% |
| d6: | 0% | d6: | 100% |
| d9: | 0% | d9: | 100% |
| d12: | 0% | d12: | 100% |
| d15: | 0% | d15: | 100% |

### Effects of Antiviral Peptide SEQ ID NO:28 on Apoptosis and Cell-Cycle Progression

### Apoptosis-Assay:

The Annexin V assay is based on the observation that soon after initiating apoptosis, most cell types translocate phosphatidylserine (PS) from the inner face of the plasma membrane to the cell surface. Once on the cell surface, PS can be easily detected by staining with a FITC conjugate of annexin V, a protein that has a strong natural affinity for PS. Externalization of PS occurs earlier than the nuclear changes associated with apoptosis, so the ApoAlert Assay detects apoptotic cells significantly earlier than do DNA-based assays. PM1 cells were cultured for 7 and 21 days in the presence of 16.3 μM antiviral peptide SEQ No. 28 or DMSO (control). Subsequently, cells were washed in PBS and assayed by FACS (FACSCalibur, Becton Dickinson) using a commercially available FITC-coupled annexin V apoptosis-kit according to the manufacturer's protocol (Bender Medsystems # BMS306FI). As shown in FIG. 5 DMSO and peptide-treated cells (21 days) revealed no difference in FACS analysis showing that the inventive peptides do not induce apoptosis.

### Cell Cycle-Analysis:

PM1 cells were cultured for 7 and 21 days in the presence of 16.3 μM antiviral peptide SEQ No. 28 or DMSO (control). Cell cycle analysis was recorded on a FACSCalibur device (Becton Dickinson) using a commercially available propidiumiodide-based DNA staining kit (CycleTest™ Plus; Becton Dickinson) according to the manufacturer's specifications. Distribution of cells in G1, G2 and S phase was similar when treated either with peptide or DMSO for 21 days. This indicates that the antiviral peptides do not induce cell cycle arrest and consequently do not inhibit cell proliferation.

### Expression of CD4 and CXCR4

The expression of CXCR4 and CD4 receptors on PM1 cells in the presence or absence of peptide SEQ ID NO. 28 was evaluated by FACS. Equal level of both receptors were found on PM1 cells either treated with DMSO or with peptide SEQ ID NO. 28. This indicates that peptide SEQ ID NO. 28 did not downregulate these receptors required for cellular entry of HIV.

### Expression of p24 and gag

Expression of p24 and gag in the presence or absence of peptide SEQ ID NO. 28 was assessed by western blot analysis using an anti-p24 antibody. As shown FIG. 7 expression of gag and p24 was strongly reduced in the presence of peptide SEQ ID NO. 28 compared to control (DMSO).

### Inhibition of Antiviral Drug-Resistant HIV-1 Isolates

PM1 cells were infected with an “omni-drug-resistant HIV-1” (recombinant HIV-1 NL4-3 in which the Protease gene and the 5′ part of the Reverse Transcriptase gene was replaced by the corresponding sequences from a omni-drug-resistant clinical isolate, resistant to all currently available anti-HIV drugs. Infected cells were incubated with peptide SEQ ID NO. 28 at a concentration of 8.1 μM, 12.2 μM, 16.3 μM or DMSO (control). The culture medium was changed every third day. Cell viability (Trypan-blue staining), cell counts and p24 levels were determined at day 6 and 12. At day 12 of infection, the virus replicated vigorously in untreated cultures. Numbers describe percent inhibition of HIV-1 replication as compared to control experiments. In addition, various different “types” of multiple anti-retroviral drug resistant HIV-1 strains were also tested.

### Inhibition of a Clinical HIV-1 Isolate

At day 10 of infection, the HIV strains replicated vigorously in untreated cultures. Numbers describe percent inhibition of HIV replication as compared to control experiments. Peripheral blood mononuclear cells (PBMCs) from HIV-1-infected patients were isolated—(after informal consent was given)—by Biocoll (Biochrom) gradient centrifugation. 1×10⁶/ml PBMCs were cultured (5 ml in a six well 32-mm plate) in presence of the test drugs or in DMSO (control diluent) in RPMI 1640 medium supplemented with 10% autologous serum and 2 mM L-Glutamine at 37° C. and 5% CO₂.

The culture medium was changed every 3 days during the experiments. Once a week, each culture was split 1:1 and 2×10⁶ feeder-PBMCs (per well) and recombinant Interleukin 2 (IL-2) [10 U/ml] (Roche) was added. Prior to addition to the cell cultures, the feeder-PBMCs, prepared from 4 healthy donors, were treated for 4 days with PHA-P and PB (2 μg/ml each per 2×10⁶ cells). At day 14, viability of the PBMCs (Trypan-blue staining), cell count and virus load (p24 antigen: Innotest HIV Antigen mAb, Innogenetics N. V., Gent, Belgium, or bDNA: HIV-1 RNA 3.0 Assay, Bayer AG, Tarrytown, N.Y., USA) was determined. No significant differences in cell viability were observed (80-90% vital cells).

### Long-Time Inhibition of a Clinical HIV-1 Isolate

PBMCs from a HIV-1-infected patient were cultured in various concentrations of peptide SEQ ID NO. 28 at a concentration of 8.1 μM, 12.2 μM, 16.3 μM or DMSO (control). Culture medium was changed every 3 days during the experiments. Once a week, each culture was split 1:1 and feeder-PBMCs (feeder cells were prepared and stimulated with PHA-P and PB) and recombinant IL-2 [10 U/ml] (Roche) were added. Every week, cell viability (Trypan-blue staining), cell counts and p24 levels were determined.

### Detection of HIV Preintegration DNA

After infection, the retroviral RNA genome is reverse transcribed into various DNA synthesis intermediates. These include full-length linear unintegrated DNA, which is transported into the nucleus for subsequent integration into the host cell genome. In addition to this linear form, covalently closed circular forms of extrachromosomal viral DNA containing 1 long term repeat (LTR) and 2 LTR junctions are found in the nucleus. Because these DNA molecules are integration-defective and only occur in de novo infected cells before a provirus is established, we refer to these molecules as preintegration DNA (PID).

A nested polymerase chain reaction (PCR) assay system for the simultaneous detection of extrachromosomal 1 LTR and 2 LTR DNA circles as described in Hauber et al. 2000: AIDS, Volume 14(16) pp 2619-2621 was used to analyse residual virus replication in PM1 cells. Total genomic DNA was isolated from ethylenediamine tetraacetic acid-blood samples and directly amplified using HIV-1-specific primer pairs for the 1 LTR and 2 LTR circular DNA (first round PCR: 5′-GCTAGAAGCACAAGAGGAGGAAGAGG-3′ and 5′-CCTGTCTGAAGG GATGGTTGTAGCTG-3′; nested PCR: 5′-GACTTACAAGGCAG CTGTAGATCTTAGC-3′ and 5′-CGAATCGTTCTAGCT CCCTGCTTGC-3′). The amplification profile involved 25 cycles of denaturation at 95° C. for 1 min, primer annealing at 58° C. for 1 min, and primer extension at 72° C. for 4 min for the first round PCR, followed by 35 cycles for the nested PCR.

### Determination of Compound Efficacy and Toxicity

Efficacy plates will be removed from the incubator, and supernatant samples (100 μl) will be removed from each well and transferred into 96-well storage plates. Pronase will be added to the supernatant samples to a final concentration of 0.75 mg/ml. Samples will be incubated at 37° C. for 30 minutes. Supernatant samples will then be treated with 1 Unit of DNase per well and incubated at 37° C. for 60 minutes to degrade DNA which has been released by dead cells. Encapsidated DNA from intact virions will not be affected by this treatment. DNase is inactivated by heating samples to 95° C. for 15 minutes. PCR reaction mixtures well be prepared from reagents provided in the PE Applied Biosystems TaqMan PCR Reagent Kit according to the manufacturer's directions. Total reaction volumes will be 50 μl. Master reaction mix will be dispensed into optical PCR tubes in a volume of 47 μl. Samples (3 μl) will be added to the reaction mix and mixed thoroughly. Reaction plates will be loaded into a PE Applied Biosystems 7700 Sequence Detector, and a run cycle will be initiated using the manufacturer's recommended PCR conditions. A standard curve prepared from known copy numbers of DNA isolated from BCBL-1 cells and amplified will be run with each plate in order to quantitate the DNA copy number in each original sample. The efficacy of the compound will be determined by comparing DNA copy numbers from test wells with those of control wells. Twenty microliters of Cell Titer Aqueous One Solution (Promega) will be added to each well of the toxicity plates. Plates will be incubated at 37° C. in a humidified CO₂ incubator for 4 hours or until sufficient color development has occurred. Plates will be read on a VMax microtiter plate reader at a wavelength of 490/650 nm. Toxicity of the test compound will be determined by comparing the optical density of test wells with that of control wells.

1. Use of at least one peptide having the amino acid sequence

| | |
|---|---|
| B-X¹-X²-X³-B′-X⁴-X⁵-X⁶-J-Tyr | (SEQ ID NO: 01) |

wherein
B and B′ are independently of each other Lys or Arg or the D-isomer thereof;
X¹-X⁶ are independently of each other norleucine or any amino acid other than a charged or polar aliphatic amino acid or the D-isomer thereof;
and J is Gly, Lys or Arg or the D-isomer thereof;
and/or pharmaceutically acceptable salts thereof for manufacturing of a medicament for prophylaxis, inhibition and/or treatment of infections mediated by viruses.

2. Use according to claim 1 wherein B and B′ are both Arg.

3. Use according to claim 1 wherein at least three of said X¹-X⁶ amino acid residues are the same non-polar aliphatic amino acid, preferably at least four are the same non-polar aliphatic amino acid, more preferably at least five are the same non-polar aliphatic amino acid, and most preferably, all are the same non-polar aliphatic amino acid.

4. Use according to claim 3 wherein said non-polar aliphatic amino acids are amino acids Val, Ile, Leu, or nL.

5. Use according to claim 1, wherein said peptide is modified at least at one terminus.

6. Use according to claim 1, wherein said peptide comprises other than (a) a naturally occurring sequence of HLA-B [alpha] 1-domain 75-84, (b) a naturally occurring sequence of the transmembrane sequence of the human T cell receptor [alpha] chain or (c) a mutated sequence of either (a) or (b) having not more than two mutations.

7. Use according to claim 1, wherein the virus is a lentivirus.

8. Use according to claim 7, wherein the virus is HIV or drug-resistant HIV or multidrug resistant strains or a strain resistant against a drug combination.

9. Use according to claim 1, wherein the disease is AIDS or HIV.

10. Use according to claim 1, wherein the peptide is selected from the group comprising: (a) Arg-Leu-Leu-Leu-Arg-Leu-Leu-Leu-Gly-Tyr (SEQ ID NO:03); (b) Arg-Val-Leu-Leu-Arg-Leu-Leu-Leu-Gly-Tyr (SEQ ID NO:04); (c) Arg-Ile-Leu-Leu-Arg-Leu-Leu-Leu-Gly-Tyr (SEQ ID NO:05); (d) Arg-Leu-Val-Leu-Arg-Leu-Leu-Leu-Gly-Tyr (SEQ ID NO: 06); (e) Arg-Leu-Ile-Leu-Arg-Leu-Leu-Leu-Gly-Tyr (SEQ ID NO:07); (f) Arg-Leu-Leu-Val-Arg-Leu-Leu-Leu-Gly-Tyr (SEQ ID NO:08); (g) Arg-Leu-Leu-Ile-Arg-Leu-Leu-Leu-Gly-Tyr (SEQ ID NO:09); (h) Arg-Leu-Leu-Leu-Arg-Val-Leu-Leu-Gly-Tyr (SEQ ID NO:10); (i) Arg-Leu-Leu-Leu-Arg-Ile-Leu-Leu-Gly-Tyr (SEQ ID NO:11); (j) Arg-Leu-Leu-Leu-Arg-Leu-Val-Leu-Gly-Tyr (SEQ ID NO:12); (k) Arg-Leu-Leu-Leu-Arg-Leu-Ile-Leu-Gly-Tyr (SEQ ID NO:13), (l) Arg-Leu-Leu-Leu-Arg-Leu-Leu-Val-Gly-Tyr (SEQ ID NO:14); (m) Arg-Leu-Leu-Leu-Arg-Leu-Leu-Ile-Gly-Tyr (SEQ ID NO:15); (n) Arg-Trp-Leu-Leu-Arg-Leu-Leu-Leu-Gly-Tyr (SEQ ID NO:16); (o) Arg-Leu-Trp-Leu-Arg-Leu-Leu-Leu-Gly-Tyr (SEQ ID NO:17); (p) Arg-Leu-Leu-Trp-Arg-Leu-Leu-Leu-Gly-Tyr (SEQ ID NO:19); (q) Arg-Leu-Leu-Leu-Arg-Trp-Leu-Leu-Gly-Tyr (SEQ ID NO:19); (r) Arg-Leu-Leu-Leu-Arg-Leu-Trp-Leu-Gly-Tyr (SEQ ID NO:20); (s) Arg-Leu-Leu-Leu-Arg-Leu-Leu-Trp-Gly-Tyr (SEQ ID NO:21); (t) Arg-Tyr-Leu-Leu-Arg-Leu-Leu-Leu-Gly-Tyr (SEQ ID NO:22); (u) Arg-Leu-Tyr-Leu-Arg-Leu-Leu-Leu-Gly-Tyr (SEQ ID NO:23); (v) Arg-Leu-Leu-Tyr-Arg-Leu-Leu-Leu-Gly-Tyr (SEQ ID NO:24); (w) Arg-Leu-Leu-Leu-Arg-Tyr-Leu-Leu-Gly-Tyr (SEQ ID NO:25); (x) Arg-Leu-Leu-Leu-Arg-Leu-Tyr-Leu-Gly-Tyr (SEQ ID NO:26); (y) Arg-Leu-Leu-Leu-Arg-Leu-Leu-Tyr-Gly-Tyr (SEQ ID NO:27); and (z) Arg-nL-nL-nL-Arg-nL-nL-nL-Gly-Tyr (SEQ ID NO:28).

11. Use according to claim 1, wherein the peptide is used in combination with a further anti-viral drug or an anti-HIV drug.

12. Use according to claim 2 wherein at least three of said X¹-X⁶ amino acid residues are the same non-polar aliphatic amino acid, preferably at least four are the same non-polar aliphatic amino acid, more preferably at least five are the same non-polar aliphatic amino acid, and most preferably, all are the same non-polar aliphatic amino acid.

13. Use according to claim 12 wherein said non-polar aliphatic amino acids are amino acids Val, Ile, Leu, or nL.


SEQUENCE LISTING (28 sequences)

SEQ ID NO: 1 - protein, 10 aa, Artificial
  synthetic peptide
XXXXXXXXXY

SEQ ID NO: 2 - protein, 10 aa, Artificial
  synthetic peptide
RXXXRXXXXY

SEQ ID NO: 3 - protein, 10 aa, Artificial
  synthetic peptide
RLLLRLLLGY

SEQ ID NO: 4 - protein, 10 aa, Artificial
  synthetic peptide
RVLLRLLLGY

SEQ ID NO: 5 - protein, 10 aa, Artificial
  synthetic peptide
RILLRLLLGY

SEQ ID NO: 6 - protein, 10 aa, Artificial
  synthetic peptide
RLVLRLLLGY

SEQ ID NO: 7 - protein, 10 aa, Artificial
  synthetic peptide
RLILRLLLGY

SEQ ID NO: 8 - protein, 10 aa, Artificial
  synthetic peptide
RLLVRLLLGY

SEQ ID NO: 9 - protein, 10 aa, Artificial
  synthetic peptide
RLLIRLLLGY

SEQ ID NO: 10 - protein, 10 aa, Artificial
  synthetic peptide
RLLLRVLLGY

SEQ ID NO: 11 - protein, 10 aa, Artificial
  synthetic peptide
RLLLRILLGY

SEQ ID NO: 12 - protein, 10 aa, Artificial
  synthetic peptide
RLLLRLVLGY

SEQ ID NO: 13 - protein, 10 aa, Artificial
  synthetic peptide
RLLLRLILGY

SEQ ID NO: 14 - protein, 10 aa, Artificial
  synthetic peptide
RLLLRLLVGY

SEQ ID NO: 15 - protein, 10 aa, Artificial
  synthetic peptide
RLLLRLLIGY

SEQ ID NO: 16 - protein, 10 aa, Artificial
  synthetic peptide
RWLLRLLLGY

SEQ ID NO: 17 - protein, 10 aa, Artificial
  synthetic peptide
RLWLRLLLGY

SEQ ID NO: 18 - protein, 10 aa, Artificial
  synthetic peptide
RLLWRLLLGY

SEQ ID NO: 19 - protein, 10 aa, Artificial
  synthetic peptide
RLLLRWLLGY

SEQ ID NO: 20 - protein, 10 aa, Artificial
  synthetic peptide
RLLLRLWLGY

SEQ ID NO: 21 - protein, 10 aa, Artificial
  synthetic peptide
RLLLRLLWGY

SEQ ID NO: 22 - protein, 10 aa, Artificial
  synthetic peptide
RYLLRLLLGY

SEQ ID NO: 23 - protein, 10 aa, Artificial
  synthetic peptide
RLYLRLLLGY

SEQ ID NO: 24 - protein, 10 aa, Artificial
  synthetic peptide
RLLYRLLLGY

SEQ ID NO: 25 - protein, 10 aa, Artificial
  synthetic peptide
RLLLRYLLGY

SEQ ID NO: 26 - protein, 10 aa, Artificial
  synthetic peptide
RLLLRLYLGY

SEQ ID NO: 27 - protein, 10 aa, Artificial
  synthetic peptide
RLLLRLLYGY

SEQ ID NO: 28 - protein, 10 aa, Artificial
  synthetic peptide
RXXXRXXXGY